(19) United States
(12) Patent Application Publication
(10) Pub. No.: US 2025/0128012 A1
(43) Pub. Date: Apr. 24, 2025

(54) CONNECTOR

(71) Applicant: Fisher & Paykel Healthcare Limited, Auckland (NZ)

(72) Inventors: Daniel Webster EVANS, Auckland (NZ); Katie-Ann Jane BUCKELS, Auckland (NZ); Kevin Blake POWELL, Auckland (NZ)

(21) Appl. No.: 18/833,279

(22) PCT Filed: Feb. 3, 2023
(86) PCT No.: PCT/NZ2023/050005

§ 371 Date: Jul. 25, 2024

Related U.S. Application Data
(60) Provisional application No. 63/306,739, filed on Feb. 4, 2022.

(51) Int. Cl. A61M 16/08; A61M 39/10; A61M 39/12

(52) U.S. Cl. CPC A61M 16/0816; A61M 39/12; A61M 2039/1033

(57) ABSTRACT

A connector for a respiratory support system having a first part, a second part, and a retention element. The first part is configured to engage a portion of a respiratory conduit and the second part is configured to engage with another component. The first and second parts together define an internal lumen for the passage of gas through the connector. The retention element defines an outer wall and the first part of the connector comprises a wall spaced inwards from the outer wall, defining a cavity therebetween to receive said portion of the respiratory conduit. The wall of the first part has one or more outwardly-projecting protrusions configured to engage said portion of the respiratory conduit. At least one distance between a surface of the protrusion(s) and an inner surface of the outer wall is less than a maximum wall thickness of the portion of the respiratory conduit.

### FIELD OF THE INVENTION

The present disclosure generally relates to a connector for a respiratory conduit, and to a tube assembly and patient interface assembly including the connector.

### BACKGROUND

In assisted breathing, respiratory gases are supplied to a patient through a patient interface via one or more flexible breathing conduits. Such therapies may include but are not limited to continuous positive airway pressure (CPAP) therapy, including for example VPAP and BiPAP systems, non-invasive ventilation (NIV) therapy, and high flow rate therapy.

Various types of respiratory patient interfaces may be used for the provision of different respiratory therapies. For example, the patient interface can be a nasal cannula, nasal mask, oral mask, or oro-nasal mask, endotracheal tube, or other known types of interfaces. The patient interface assembly for the individual patient must be fluidly coupled to the other components of the respiratory therapy system to enable the provision of humidified respiratory gases to a patient. This coupling may be facilitated through a connector that connects with a conduit or component of the patient interface assembly and to a conduit or other component of the respiratory system.

Accidental detachment and/or disengagement of one of the conduits or components from the connector is undesirable, but can happen under sufficient axial loading, for example when a tensile force or another force having a tensile axial component is applied to a connector. The applied loading may cause an attached conduit or component to pull off or out of the connector. In some examples a conduit may disconnect from a respective connector upon deflecting or stretching of the conduit wall, enabling the conduit to slide over engagement features of the connector intended to hold the conduit in place.

In the specification where reference has been made to patent specifications, other external documents, or other sources of information, this is generally for the purpose of providing a context for discussing the features of the disclosure. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

In a first aspect, the present disclosure relates to a connector for a respiratory support system, comprising a first part, a second part, and a retention element. The first part is configured to engage a portion of a respiratory conduit and the second part is configured to engage with another component. Together, the first and second parts define an internal lumen for the passage of gas through the first and second parts. The retention element defines an outer wall. The first part of the connector comprises an wall spaced inwards from the outer wall, thereby defining a cavity between the inner and outer walls to receive said portion of the respiratory conduit, the wall of the first part having one or more outwardly-projecting protrusions configured to engage said portion of the respiratory conduit; and wherein at least one distance between a surface of the protrusion and an inner surface of the outer wall is less than a maximum wall thickness of the portion of the respiratory conduit.

The closest distance between the, or each, protrusion and the inner surface of the outer wall may be less than a bead size of the conduit. The closest distance may be a measured in a radial direction.

In an embodiment, the connector is configured to locate the maximum wall thickness of the portion of the respiratory conduit between the outwardly-projecting protrusion(s).

In an embodiment, the first part of the connector is configured to engage with another connector.

In an embodiment, at least one distance between an outer surface of the wall of the first part and an inner surface of the outer wall is greater than the maximum wall thickness of the portion of the respiratory conduit.

In an embodiment, at least one distance between a surface of the, or each, protrusion and an inner surface of the outer wall is greater than the maximum wall thickness of the portion of the respiratory conduit.

In an embodiment, the maximum wall thickness of the respiratory conduit corresponds to the thickness of a bead of the conduit. The closest distance between the, or each, protrusion and the inner surface of the outer wall may be at least 1% less than the maximum wall thickness or bead size, preferably at least 10% or 20% less. In an embodiment, the closest distance between the, or each, protrusion and the inner surface of the outer wall about 50% or more less than the maximum wall thickness or bead size, for example about 55% less than the bead thickness.

In an embodiment, the closest distance between the, or each, protrusion and the inner surface of the outer wall is greater than a minimum wall thickness of the respiratory conduit.

The minimum wall thickness of the conduit may be a film thickness of between about 20 μm and about 120 μm. In one embodiment, the minimum wall thickness of the conduit is about 50 μm. The minimum wall thickness of the respiratory conduit may correspond to the thickness of the film between beads. The closest distance between the, or each, protrusion and the inner surface of the outer wall may be at least 1% more than the minimum wall thickness, preferably at least 10% or 20% more than the minimum wall thickness. In some embodiments, the closest distance between the, or each, protrusion and the inner surface of the outer wall may be many times greater than the film thickness.

In an embodiment, when the conduit is connected with the second part, the conduit wall is spaced inwards from the inner surface of the outer wall.

In an embodiment, the protrusion(s) forms a plurality of lobes aligned along a substantially helical path and each lobe comprises a leading portion at a leading end of the lobe and a trailing portion at a trailing end of the lobe, the trailing portion having a steeper gradient with respect to a surface of the first part than the leading portion.

In an embodiment, during assembly of the first part of the connector with the respiratory conduit, the leading portion of each lobe engages the wall of the conduit before the respective trailing portion.

The lobes may be formed by a single helical protrusion or may be formed by a plurality of discrete protrusions.

In an embodiment, the trailing portion of one lobe may be spaced from, may contact, or may be contiguous with leading portion of an adjacent lobe. In some embodiments, other projections or features may be present between lobes.

A maximum height of each lobe may occur at a point intermediate the respective leading and trailing ends. In an embodiment, the maximum height of each lobe is at a point closer to the trailing end of said lobe than the leading end of the lobe. The maximum height of each lobe may be substantially the same for each lobe or may vary between lobes. For example, gradually increase from a first (leading) lobe to a trailing lobe.

In an embodiment, the, or each, protrusion has a generally smooth contour. For example, the side edges of the projection(s) may be rounded or otherwise shaped so that there are no sharp corners on the lobe, to thereby minimise stress concentrations in the respiratory conduit. In an embodiment, the side edges of the lobes are filleted.

In an embodiment, the leading end of each lobe, the trailing end of each lobe, and an axis of the connector form an angle of about 90 degrees. In alternative embodiments, the lobes may be shorter or longer with respect to the connector first part. The leading end of each lobe, the trailing end of each lobe, and an axis of the connector may form an angle that is more than or less than 90 degrees, for example, between about 30 degrees and about 360 degrees, preferably between about 45 and 120 degrees.

In an embodiment, four lobes are provided for each coil of the helical path. Alternatively, more or fewer than four lobes may be provided for each coil, for example, between one and eight lobes may be provided for each coil of the helical path. In some embodiments, the number of lobes per coil may not be a whole number, for example, 2.5 or 4.3 lobes per rotation.

In an embodiment, the helical path has a pitch that is substantially the same as the pitch of a helical feature on the respiratory conduit.

In an embodiment, the one or more protrusions are configured such that a torque required to wind the conduit onto the connector is less than a torque required to unwind the conduit from the connector.

In an embodiment, the first part of the connector is rotatably connected to the second part of the connector. Alternatively, the first and second parts may be fixed relative to each other. The first and second parts of the connector may be separate, connected components, or may be integrally formed.

In an embodiment, a first end of the retention element is engaged with the connector. A first end of the retention element may be engaged with the first part or second part of the connector. The retention component may be integral with the second part of the body.

In an embodiment, the retention element comprises a sleeve. For example, the retention element may have a generally cylindrical form. Optionally, the retention element may flare outward at one end to assist with assembly.

The retention element may be co-axial with the second part of the connector.

In an embodiment, the at least one distance between a surface of the protrusion and an inner surface of the outer wall is selected to inhibit or prevent disconnection of the respiratory conduit from the connector due to a generally axial force.

In a second aspect, the present disclosure relates to a connector for a respiratory support system, comprising a first part configured to engage the wall of a portion of a respiratory conduit, a second part configured to engage with another component, and a retention element to prevent pull-off of the conduit from the connector. Together, the first and second parts define an internal lumen for the passage of gas through the first and second parts. The first part of the connector comprises a wall spaced inwards from the retention element, thereby defining a cavity between the wall and the retention element to receive said portion of the respiratory conduit. The first part of the connector comprises an engagement region with or more protrusions projecting outwardly into said cavity, and configured to engage said portion of the respiratory conduit. At least one distance between a surface of the protrusion and an inner surface of the retention element is less than a maximum wall thickness of the respiratory conduit.

In a third aspect, the present disclosure relates to a connector for a respiratory support system, the connector having an internal lumen for the passage of gas therethrough. A first part of the connector is configured to engage a wall of a portion of a respiratory conduit and a second part of the connector is configured to engage with another component. The connector comprises an inner wall and an outer wall, with an annular cavity defined therebetween to receive the wall of the respiratory conduit. The inner wall comprises an engagement region with or more protrusions projecting outwardly into said cavity, configured to engage said wall of the respiratory conduit. At least one distance between a surface of the protrusion and an inner surface of the outer wall is less than a maximum thickness of the wall of the respiratory conduit.

In a fourth aspect, the present disclosure relates to an interface assembly for a respiratory support system comprising a patient interface, a respiratory conduit connected to the patient interface, and a connector as describe above according to any of the first three aspects. The first part of the connector engages an end portion of the respiratory conduit.

In an embodiment, a wall of the conduit comprises a flexible film. The flexible film may be breathable. The film of the conduit may have a wall thickness of between about 20 μm and about 120 μm. In one embodiment, the film thickness of the conduit is about 50 μm. The film may have a width between about 6 mm and about 10 mm, for example about 8 mm.

In an embodiment, a wall of the conduit comprises a helical bead. The bead may have a height of between about 0.5 mm and about 3 mm, for example a height of about 1 mm. The bead may have a width of between about 1 mm and about 2 mm, for example a width of about 2 mm.

The helical bead may have a pitch length between about 2.6 mm and about 5 mm. In one embodiment, the helical bead has a pitch of 4.5 mm.

In an embodiment, the conduit has an inner diameter of about 12 mm and an outer diameter of about 14 mm. In alternative embodiments, the inner diameter may be between about 10 mm and about 14 mm; and the outer diameter may be between about 12 mm and about 16 mm.

The respiratory conduit may be of any suitable length. For example, between about 200 mm and about 500 mm, preferably between about 300 mm and about 450 mm. In one embodiment, the conduit has a length of about 370 mm.

The interface assembly may comprise asymmetrical delivery elements configured to cause an asymmetrical flow at the patient.

In a fifth aspect, the present disclosure relates to a connector for a respiratory support system, comprising a first part configured to engage a portion of a respiratory conduit, and second part configured to engage with another connector, and a retention element. The first and second parts together define an internal lumen for the passage of gas through the first and second parts, and the retention element defines an outer wall. The first part comprises a wall spaced inwards from the outer wall, forming an inner wall of the connector and thereby defining a cavity between the inner and outer walls to receive said portion of the respiratory conduit, the inner wall having a protrusion configured to engage said portion of the respiratory conduit. A distance between a surface of the protrusion and an inner surface of the outer wall is selected to inhibit or prevent disconnection of the respiratory conduit from the connector due to a generally axial force.

The distance between a surface of the protrusion and an inner surface of the outer wall is selected such that, upon application of an axial force to the conduit that is above a threshold force, the wall of the conduit will tear while the second part of the connector remains engaged with the conduit.

At least one distance between a surface of the protrusion and an inner surface of the outer wall may be less than a maximum wall thickness of the respiratory conduit.

The closest distance between the protrusion and the inner surface of the outer well may be less than a bead size of the conduit. The closest distance may be a measured in a radial direction.

In an embodiment, at least one distance between an outer surface of the inner wall and an inner surface of the outer wall is greater than a maximum wall thickness of the respiratory conduit.

In an embodiment, at least one distance between a surface of the, or each, protrusion and an inner surface of the outer wall is greater than a maximum wall thickness of the respiratory conduit.

In an embodiment, the maximum wall thickness of the respiratory conduit corresponds to the thickness of a bead of the conduit. The closest distance between the, or each, protrusion and the inner surface of the outer wall may be at least 1% less than the maximum wall thickness or bead size, preferably at least 10% or 20% less. In an embodiment, the closest distance between the, or each, protrusion and the inner surface of the outer wall about 50% or more less than the maximum wall thickness or bead size, for example about 55% less than the bead thickness.

In an embodiment, the closest distance between the, or each, protrusion and the inner surface of the outer wall is greater than a minimum wall thickness of the respiratory conduit.

The minimum wall thickness of the conduit may be a film thickness of between about 20 μm and about 120 μm. In one embodiment, the minimum wall thickness of the conduit is about 50 μm. The minimum wall thickness of the respiratory conduit may correspond to the thickness of the film between beads. The closest distance between the, or each, protrusion and the inner surface of the outer wall may be at least 1% more than the minimum wall thickness, preferably at least 10% or 20% more than the minimum wall thickness. In some embodiments, The closest distance between the, or each, protrusion and the inner surface of the outer wall may be many times greater than the film thickness.

In an embodiment, when the conduit is connected with the second part, the conduit wall is spaced inwards from the inner surface of the outer wall.

In an embodiment, the first part of the connector comprises one or more protrusions that form a plurality of lobes aligned along a substantially helical path, each lobe comprising a leading portion at a leading end of the lobe and a trailing portion at a trailing end of the lobe, the trailing portion having a steeper gradient with respect to a surface of the first part than the leading portion. During assembly of the first part of the connector with the respiratory conduit, the leading portion of each lobe may engage the wall of the conduit before the respective trailing portion.

The one or more protrusions may form a continuous or discontinuous thread. The lobes may be formed by a single helical protrusion or the lobes may be formed by a plurality of discrete protrusions.

In an embodiment, the height of each lobe, from the base of the lobe to the top of the lobe, varies along the lobe from the leading portion to the trailing portion.

In an embodiment, the trailing portion of one lobe may be spaced from, may contact, or may be contiguous with leading portion of an adjacent lobe. In some embodiments, other projections or features may be present between lobes.

A maximum height of each lobe may occur at a point intermediate the respective leading and trailing ends. In an embodiment, the maximum height of each lobe is at a point closer to the trailing end of said lobe than the leading end of the lobe. The maximum height of each lobe may be substantially the same for each lobe or may vary between lobes. For example, gradually increase from a first (leading) lobe to a trailing lobe.

A maximum height of each lobe may be at a point closer to the trailing end of said lobe than the leading end of the lobe.

In an embodiment, the, or each, protrusion has a generally smooth contour. For example, the side edges of the projection(s) may be rounded or otherwise shaped so that there are no sharp corners on the lobe, to thereby minimise stress concentrations in the respiratory conduit. In an embodiment, the side edges of the lobes are filleted.

In an embodiment, the leading end of each lobe, the trailing end of each lobe, and an axis of the connector form an angle of about 90 degrees. In alternative embodiments, the lobes may be shorter or longer with respect to the connector first part. The leading end of each lobe, the trailing end of each lobe, and an axis of the connector may form an angle that is more than or less than 90 degrees, for example, between about 30 degrees and about 360 degrees, preferably between about 45 and 120 degrees.

In an embodiment, four lobes are provided for each coil of the helical path. Alternatively, more or fewer than four lobes may be provided for each coil, for example, between one and eight lobes may be provided for each coil of the helical path. In some embodiments, the number of lobes per coil may not be a whole number, for example, 2.5 or 4.3 lobes per rotation.

In an embodiment, the helical path has a pitch that is substantially the same as the pitch of a helical feature on the respiratory conduit.

In an embodiment, the one or more protrusions are configured such that a torque required to wind the conduit onto the connector is less than a torque required to unwind the conduit from the connector.

In an embodiment, a first end of the retention element is engaged with the connector. A first end of the retention element may be engaged with the first part or second part of the connector. The retention component may be integral with the second part of the body.

In an embodiment, the retention element comprises a sleeve. For example, the retention element may have a generally cylindrical form. Optionally, the retention element may flare outward at one end to assist with assembly.

The retention element may be co-axial with the second part of the connector.

In a sixth aspect, the present disclosure relates to a connector for a respiratory support system, comprising a first part configured to engage a portion of a respiratory conduit, and a second part configured to engage with another connector, the first and second parts together defining an internal lumen for the passage of gas through the first and second parts. The first part comprises one or more protrusions that form a plurality of lobes aligned along a substantially helical path. Each lobe comprises a leading portion at a leading end of the lobe and a trailing portion at a trailing end of the lobe, the trailing portion having a steeper gradient with respect to a surface of the first part than the leading portion.

During assembly of the first part of the connector with the respiratory conduit, the leading portion of each, lobe may engage the wall of the conduit before the respective trailing portion.

The one or more protrusions may form a continuous or discontinuous thread. The lobes may be formed by a single helical protrusion or the lobes may be formed by a plurality of discrete protrusions.

In an embodiment, the height of each lobe, from the base of the lobe to the top of the lobe, varies along the lobe from the leading portion to the trailing portion.

In an embodiment, the trailing portion of one lobe may be spaced from, may contact, or may be contiguous with leading portion of an adjacent lobe. In some embodiments, other projections or features may be present between lobes.

A maximum height of each lobe may occur at a point intermediate the respective leading and trailing ends. In an embodiment, the maximum height of each lobe is at a point closer to the trailing end of said lobe than the leading end of the lobe. The maximum height of each lobe may be substantially the same for each lobe or may vary between lobes. For example, gradually increase from a first (leading) lobe to a trailing lobe.

A maximum height of each lobe may be at a point closer to the trailing end of said lobe than the leading end of the lobe.

In an embodiment, the, or each, protrusion has a generally smooth contour. For example, the side edges of the projection(s) may be rounded or otherwise shaped so that there are no sharp corners on the lobe, to thereby minimise stress concentrations in the respiratory conduit. In an embodiment, the side edges of the lobes are filleted.

In an embodiment, the leading end of each lobe, the trailing end of each lobe, and an axis of the connector form an angle of about 90 degrees. In alternative embodiments, the lobes may be shorter or longer with respect to the connector first part. The leading end of each lobe, the trailing end of each lobe, and an axis of the connector may form an angle that is more than or less than 90 degrees, for example, between about 30 degrees and about 360 degrees, preferably between about 45 and 120 degrees.

In an embodiment, four lobes are provided for each coil of the helical path. Alternatively, more or fewer than four lobes may be provided for each coil, for example, between one and eight lobes may be provided for each coil of the helical path. In some embodiments, the number of lobes per coil may not be a whole number, for example, 2.5 or 4.3 lobes per rotation.

In an embodiment, the helical path has a pitch that is substantially the same as the pitch of a helical feature on the respiratory conduit.

In an embodiment, the one or more protrusions are configured such that a torque required to wind the conduit onto the connector is less than a torque required to unwind the conduit from the connector.

When the connector is engaged with the conduit, the tension in the wall of the conduit may vary relative to the lobes. Maximum tension may typically occur in the film of the conduit wall, proximal the maximum height points of the lobe.

The connector may comprise a retention element to prevent pull-off of the conduit from the connector. The retention element may define an outer wall that is spaced outwards from the first part of the connector and the lobes thereon. In an embodiment, in a seated position of the conduit within the connector, the conduit wall is spaced inwards from the inner surface of the outer wall.

In an embodiment, a distance between a maximum height of the lobes and an inner surface of the outer wall is selected to inhibit or prevent disconnection of the respiratory conduit from the connector under a generally axial force.

In an embodiment, the distance between a surface of the protrusion and an inner surface of the outer wall is selected such that, upon application of an axial force to the conduit that is above a threshold force, the wall of the conduit will tear while the second part of the connector remains engaged with the conduit.

In an embodiment, at least one distance between a surface of the protrusion and an inner surface of the outer wall is less than a maximum wall thickness of the respiratory conduit.

The closest distance between the protrusion and the inner surface of the outer well may be less than a bead size of the conduit. The closest distance may be a measured in a radial direction.

In an embodiment, at least one distance between an outer surface of the inner wall and an inner surface of the outer wall is greater than a maximum wall thickness of the respiratory conduit.

In an embodiment, at least one distance between a surface of the, or each, protrusion and an inner surface of the outer wall is greater than a maximum wall thickness of the respiratory conduit.

In an embodiment, the maximum wall thickness of the respiratory conduit corresponds to the thickness of a bead of the conduit. The closest distance between the, or each, protrusion and the inner surface of the outer wall may be at least 1% less than the maximum wall thickness or bead size, preferably at least 10% or 20% less. In an embodiment, the closest distance between the, or each, protrusion and the inner surface of the outer wall about 50% or more less than the maximum wall thickness or bead size, for example about 55% less than the bead thickness.

In an embodiment, the closest distance between the, or each, protrusion and the inner surface of the outer wall is greater than a minimum wall thickness of the respiratory conduit.

The minimum wall thickness of the conduit may be a film thickness of between about 20 μm and about 120 μm. In one embodiment, the minimum wall thickness of the conduit is about 50 μm. The minimum wall thickness of the respiratory conduit may correspond to the thickness of the film between beads. The closest distance between the, or each, protrusion and the inner surface of the outer wall may be at least 1% more than the minimum wall thickness, preferably at least 10% or 20% more than the minimum wall thickness. In some embodiments, the closest distance between the, or each, protrusion and the inner surface of the outer wall may be many times greater than the film thickness.

In an embodiment, the first part of the connector is rotatably connected to the second part. Alternatively, the first and second parts may be fixed relative to each other. The first and second parts of the connector may be separate, connected components, or may be integrally formed.

In an embodiment, the retention element comprises a sleeve. For example, the retention element may have a generally cylindrical form. Optionally, the retention element may flare outward at one end to assist with assembly.

The retention element may be co-axial with the second part of the connector.

In a seventh aspect, the present disclosure relates to a patient interface assembly for a respiratory support system, comprising: a patient interface; a respiratory conduit connected to the patient interface; and a connector as described above in relation to the sixth aspect. The first part of the connector engages an end portion of the respiratory conduit.

In an embodiment, a wall of the conduit comprises a flexible film. The flexible film may be breathable. The film of the conduit may have a wall thickness of between about 20 μm and about 120 μm. In one embodiment, the film thickness of the conduit is about 50 μm. The film may have a width between about 6 mm and about 10 mm, for example about 8 mm.

The lobes of the connector may be configured to engage the flexible film. Each lobe may contact the wall or film along a portion of the lobe or along substantially all of the lobe. Engagement of the lobes with the conduit wall may cause deflection of the wall around the lobe.

In an embodiment, a wall of the conduit comprises a helical bead. The bead may have a height of between about 0.5 mm and about 3 mm, for example a height of about 1 mm. The bead may have a width of between about 1 mm and about 2 mm, for example a width of about 2 mm.

In an embodiment, the connector is configured such that bead is seated between the successive coils of the helical path formed buy the projection(s). The helical bead may have a pitch length between about 2.6 mm and about 5 mm. In one embodiment, the helical bead has a pitch of 4.5 mm.

In an embodiment, the conduit has an inner diameter of about 12 mm and an outer diameter of about 14 mm. In alternative embodiments, the inner diameter may be between about 10 mm and about 14 mm; and the outer diameter may be between about 12 mm and about 16 mm.

The respiratory conduit may be of any suitable length. For example, between about 200 mm and about 500 mm, preferably between about 300 mm and about 450 mm. In one embodiment, the conduit has a length of about 370 mm.

The interface assembly may comprise asymmetrical delivery elements configured to cause an asymmetrical flow at the patient.

In an eighth aspect, the present disclosure relates to a connector for a respiratory support system, comprising a first part configured to engage a portion of a respiratory conduit, and a second part configured to engage with another connector. The first and second parts together define an internal lumen for the passage of gas through the first and second parts. The first part comprises one or more protrusions shaped to engage an internal surface of a wall of said portion of the respiratory conduit and the protrusions are shaped to minimise stress concentrations in regions of the conduit wall proximate the protrusions upon engagement of the connector with the conduit.

In an embodiment, the protrusion(s) forms a plurality of lobes aligned along a substantially helical path, and each lobe comprises a leading portion at a leading end of the lobe and a trailing portion at a trailing end of the lobe, the trailing portion having a steeper gradient with respect to a surface of the first part than the leading portion.

In an embodiment, during assembly of the first part of the connector with the respiratory conduit, the leading portion of each, lobe engages the wall of the conduit before the respective trailing portion.

The lobes may be formed by a single helical protrusion or the lobes may be formed by a plurality of discrete protrusions.

In an embodiment, the height of each lobe, from the base of the lobe to the top of the lobe, varies along the lobe from the leading portion to the trailing portion.

In an embodiment, the trailing portion of one lobe may be spaced from, may contact, or may be contiguous with leading portion of an adjacent lobe. In some embodiments, other projections or features may be present between lobes.

A maximum height of each lobe may occur at a point intermediate the respective leading and trailing ends. In an embodiment, the maximum height of each lobe is at a point closer to the trailing end of said lobe than the leading end of the lobe. The maximum height of each lobe may be substantially the same for each lobe or may vary between lobes. For example, gradually increase from a first (leading) lobe to a trailing lobe.

A maximum height of each lobe may be at a point closer to the trailing end of said lobe than the leading end of the lobe.

In an embodiment, the, or each, protrusion has a generally smooth contour. For example, the side edges of the projection(s) may be rounded or otherwise shaped so that there are no sharp corners on the lobe, to thereby minimise stress concentrations in the respiratory conduit. In an embodiment, the side edges of the lobes are filleted.

In an embodiment, the leading end of each lobe, the trailing end of each lobe, and an axis of the connector form an angle of about 90 degrees. In alternative embodiments, the lobes may be shorter or longer with respect to the connector first part. The leading end of each lobe, the trailing end of each lobe, and an axis of the connector may form an angle that is more than or less than 90 degrees, for example, between about 30 degrees and about 360 degrees, preferably between about 45 and 120 degrees.

In an embodiment, four lobes are provided for each coil of the helical path. Alternatively, more or fewer than four lobes may be provided for each coil, for example, between one and eight lobes may be provided for each coil of the helical path. In some embodiments, the number of lobes per coil may not be a whole number, for example, 2.5 or 4.3 lobes per rotation.

In an embodiment, the helical path has a pitch that is substantially the same as the pitch of a helical feature on the respiratory conduit.

In an embodiment, the one or more protrusions are configured such that a torque required to wind the conduit onto the connector is less than a torque required to unwind the conduit from the connector.

In a ninth aspect, the present disclosure relates to a patient interface assembly for a respiratory support system comprising a patient interface, a respiratory conduit connected to the patient interface and a connector for a respiratory support system. The connector comprises a first part configured to engage an end portion of the respiratory conduit, a second part configured to engage with another component, and a retention element defining an outer wall. Together, the first and second parts of the connector define an internal lumen for the passage of gas through the first and second parts. The first part of the connector comprises a wall that is spaced inwards from the outer wall, thereby defining a cavity between said wall and the outer wall to receive the end portion of the respiratory conduit, the wall of the first part having one or more outwardly-projecting protrusions configured to engage the respiratory conduit. At least one distance between a surface of the protrusion(s) and an inner surface of the outer wall is less than a maximum wall thickness of the end portion of the respiratory conduit.

In an embodiment, the closest distance between the, or each, protrusion and the inner surface of the outer wall is less than a bead size of the conduit.

In an embodiment, at least one distance between an outer surface of the wall of the first part and an inner surface of the outer wall is greater than the maximum wall thickness of the portion of the respiratory conduit.

In an embodiment, at least one distance between a surface of the, or each, protrusion and an inner surface of the outer wall is greater than the maximum wall thickness of the portion of the respiratory conduit.

In an embodiment, the closest distance between the, or each, protrusion and the inner surface of the outer wall is greater than a minimum wall thickness of the respiratory conduit.

In an embodiment, the protrusion(s) forms a plurality of lobes aligned along a substantially helical path. Each lobe may comprise a leading portion at a leading end of the lobe and a trailing portion at a trailing end of the lobe. The trailing portion may have a steeper gradient with respect to a surface of the first part than the leading portion.

In an embodiment, during assembly of the first part of the connector with the respiratory conduit, the leading portion of each lobe engages the wall of the conduit before the respective trailing portion.

In an embodiment, the lobes are formed by a single helical protrusion. Alternatively, the lobes may be formed by a plurality of discrete protrusions.

In an embodiment, a maximum height of each lobe is at a point closer to the trailing end of said lobe than the leading end of the lobe.

In an embodiment, the leading end of each lobe, the trailing end of each lobe, and an axis of the connector form an angle of about 90 degrees.

In an embodiment, four lobes are provided for each coil of the helical path.

In an embodiment, the helical path has a pitch that is substantially the same as the pitch of a helical feature on the respiratory conduit.

In an embodiment, the one or more protrusions are configured such that a torque required to wind the conduit onto the connector is less than a torque required to unwind the conduit from the connector.

In an embodiment, the first part of the connector is rotatably connected to the second part of the connector.

In an embodiment, a first end of the retention element is engaged with the connector.

In an embodiment, the retention element comprises a sleeve.

In an embodiment, a wall of the conduit comprises a flexible film.

In an embodiment, the flexible film is breathable.

In an embodiment, a wall of the conduit comprises a helical bead.

In an embodiment, the at least one distance between a surface of the protrusion and an inner surface of the outer wall is selected to inhibit or prevent disconnection of the respiratory conduit from the connector due to a generally axial force.

The interface assembly may comprise asymmetrical delivery elements configured to cause an asymmetrical flow at the patient.

This invention may also be said broadly to consist in the parts, elements and features referred to or indicated in the specification of the application, individually or collectively, and any or all combinations of any two or more said parts, elements or features. Where specific integers are mentioned herein which have known equivalents in the art to which this invention relates, such known equivalents are deemed to be incorporated herein as if individually described.

The term ‘comprising’ as used in this specification and claims means ‘consisting at least in part of’. When interpreting statements in this specification and claims that include the term ‘comprising’, other features besides those prefaced by this term can also be present. Related terms such as ‘comprise’ and ‘comprised’ are to be interpreted in a similar manner.

It is intended that reference to a range of numbers disclosed herein (for example, 1 to 10) also incorporates reference to all rational numbers within that range and any range of rational numbers within that range (for example, 1 to 6, 1.5 to 5.5 and 3.1 to 10). Therefore, all sub-ranges of all ranges expressly disclosed herein are hereby expressly disclosed.

As used herein the term ‘(s)’ following a noun means the plural and/or singular form of that noun. As used herein the term ‘and/or’ means ‘and’ or ‘or’, or where the context allows, both.

### BRIEF DESCRIPTION OF THE DRAWINGS

Embodiments will now be described by way of example only and with reference to the accompanying drawings in which:

FIG. 1 is a schematic of a first embodiment respiratory support system for supplying humidified gases to a patient;

FIG. 2 is an illustrative perspective of a second embodiment respiratory support system, for supplying humidified gases to a patient via a nasal cannula;

FIG. 3 is an illustrative perspective view showing an interface for the supply of gases to a patient via a tracheal tube;

FIG. 4 is an exploded perspective view of a patient interface assembly from the system illustrated in FIG. 2, including a length of respiratory conduit and a connector;

FIG. 5 is an exploded perspective view of the patient interface assembly from the system illustrated in FIG. 3, including a length of respiratory conduit and the connector as also illustrated in FIG. 4;

FIG. 6 is a schematic cross-sectional view of the wall along a portion of an exemplary respiratory tube, the tube having a helical bead with a flat base and rounded outer side;

FIG. 7 is a schematic cross-sectional view of the wall along a portion of an exemplary respiratory tube, the tube having a helical bead with a generally oval cross-section;

FIG. 8 is a cross section view of a first embodiment connector connected to a respiratory conduit;

FIG. 9 is a cross section view of a second embodiment connector connected to a respiratory conduit;

FIG. 10 a front perspective view of a first part of the connector shown in FIG. 8;

FIG. 11 a rear perspective view of a first part of the connector shown in FIG. 8;

FIG. 12 is a front elevation view of the first part of the connector shown in FIGS. 8, 10 and 11;

FIG. 13 is a side elevation view of the first part of the connector shown in FIGS. 8 and 10 to 12;

FIG. 14 is an end view of the first part of the connector shown in FIGS. 10 to 13;

FIG. 15 is an illustrative end view showing the shape and position of the lobes on the connector part of FIGS. 10 to 14;

FIG. 16 is a perspective view showing a second part of the connector shown in FIG. 8, and including the retention element;

FIG. 17 is a cross-sectional perspective view of the second connector part shown in FIG. 16, taken through a mid-plane of the connector

FIG. 18 is a front left perspective view of an example patent interface comprising a nasal interface with asymmetrical nasal delivery elements.

FIG. 19 shows the nasal interface of FIG. 18, where FIG. 19 (a) is a top view, FIG. 19 (b) is a front view, and FIG. 19 (c) is a bottom view.

FIG. 20 is an example patient interface connected to a respiratory conduit and connector assembly;

FIG. 21 is a perspective view of the respiratory conduit and connector assembly of FIG. 20;

FIG. 22 is an exploded view of the respiratory conduit and connector assembly of FIG. 21;

FIG. 23 is a perspective view of a swivel connector of the assembly of FIG. 21;

FIG. 24 is an exploded perspective view of the swivel connector of FIG. 23;

FIG. 25 is an exploded side view of the swivel connector of FIG. 23; and

FIG. 26 is a cross-sectional side view of the second connector and swivel connector of FIG. 23.

### DETAILED DESCRIPTION

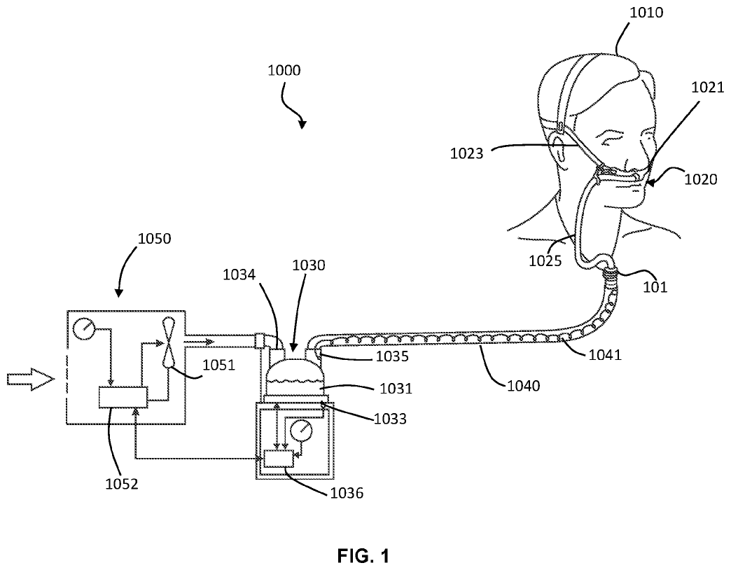

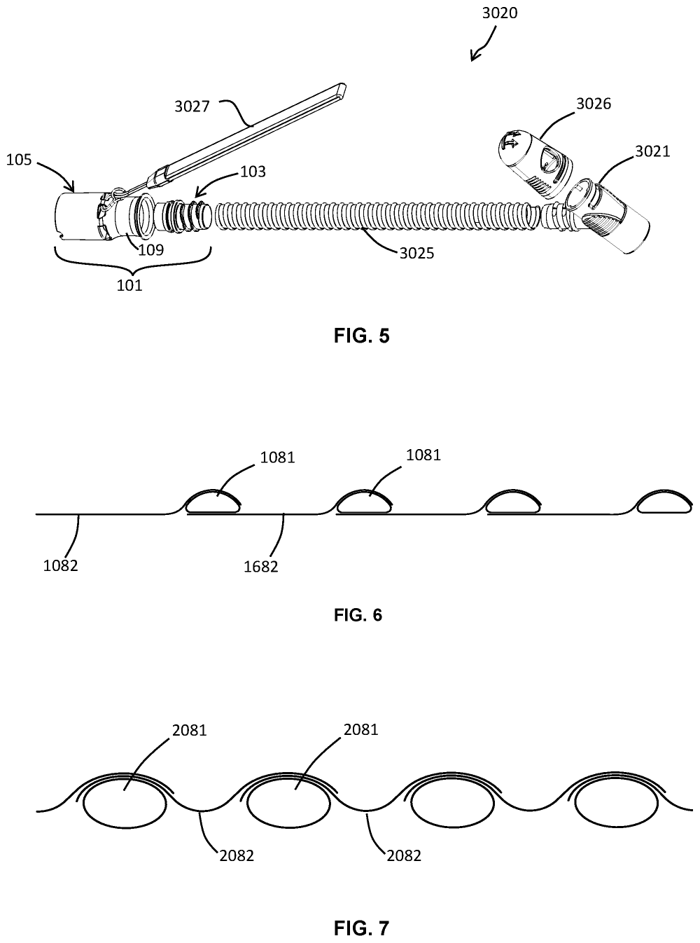

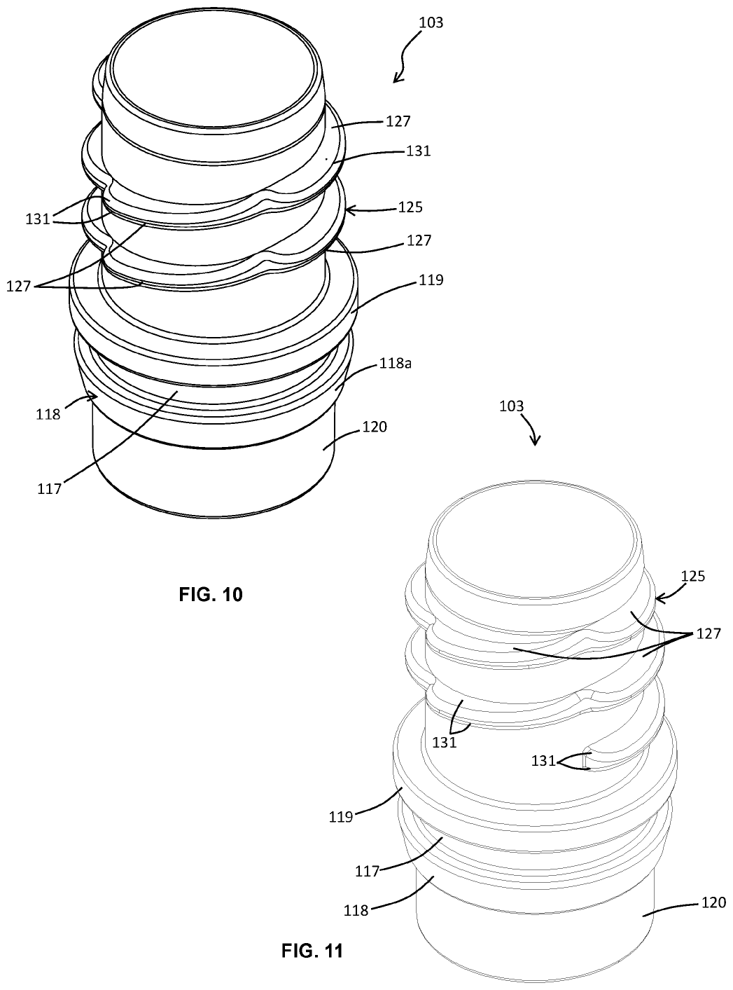

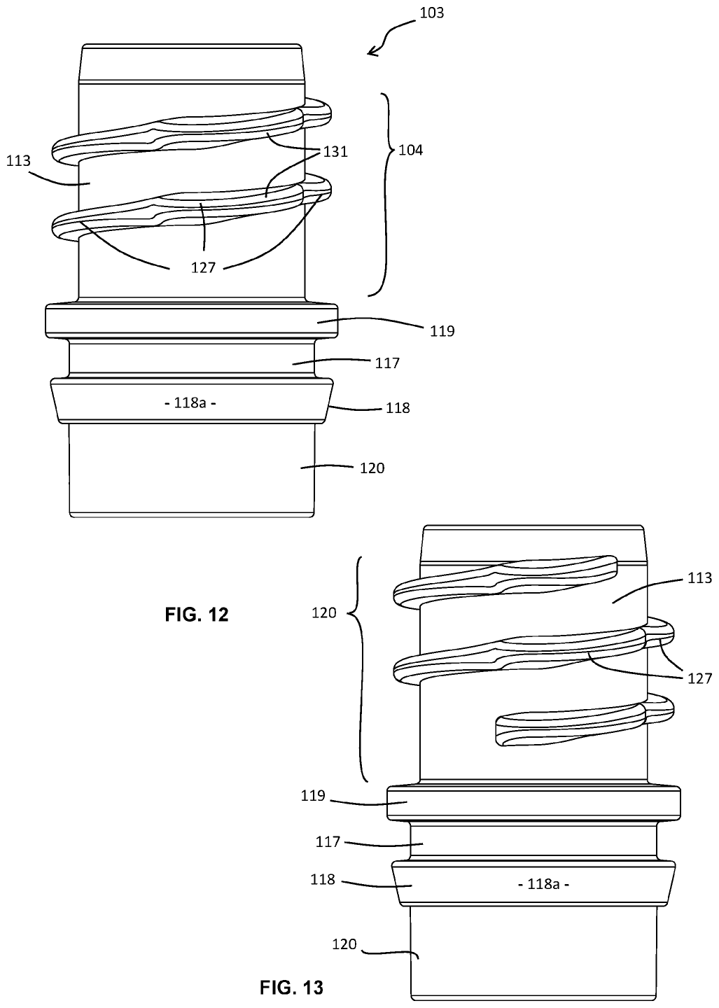

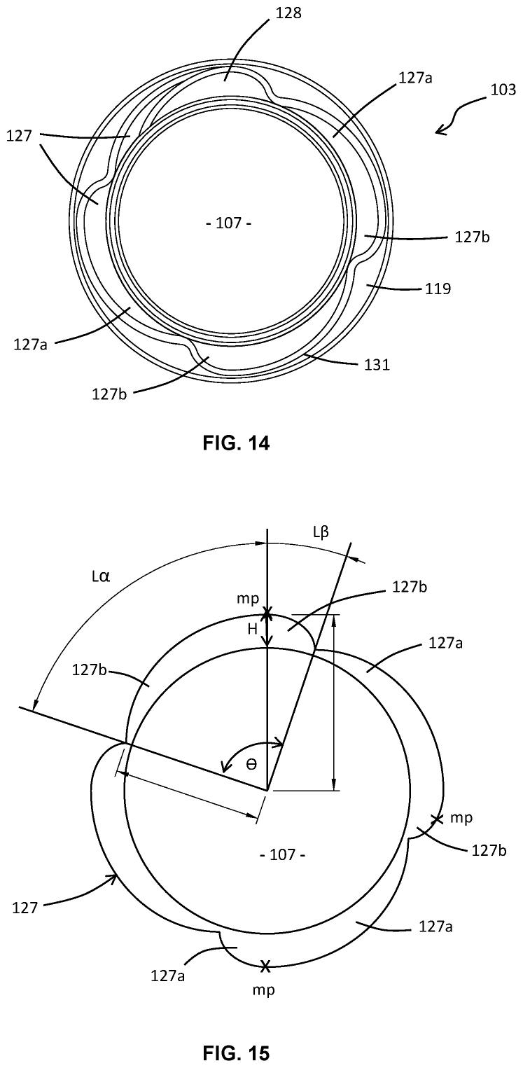

Various embodiments and methods of manufacture will now be described with reference to FIGS. 1 to 17.

Directional terminology used in the following description is for ease of description and reference only, it is not intended to be limiting. For example, the terms ‘front’, ‘rear’, ‘upper’, ‘lower’, and other related terms refer to the location of a part or portion of a respiratory mask relative to a user when the user is wearing the respiratory mask. In this specification, ‘rear’ refers to a location that is proximal to the user (when the mask is in use) and ‘front’ refers to a location that is distal to the user by comparison. The terms ‘upper’ and ‘lower’ refer to the location of a part or component of a mask relative to the rest of the mask when the mask is in use and the user is sitting in an upright position.

### Respiratory System

FIG. 1 shows an example respiratory system 1000 in which embodiments of the connector 101 described herein can be used. In the illustrated arrangement, a patient 1010 is receiving humidified and pressurised gases through a patient interface assembly 1020 coupled to a respiratory conduit 1040 that in turn is connected to a humidifier 1030 (including humidification chamber 1031) that is supplied with gases from a gases source 1050.

The gases source 1050 may comprise a blower 1051 which draws air or other gases through the blower inlet. Supplementary oxygen (not shown) may also be supplied to the blower 1023. The blower 1051 may be controlled, for example by an electronic controller 1052 in response to inputs from system sensors and/or from a controller for another system component such as the humidifier 1036, and/or in response to user input values. In an alternative embodiment, the gases source 1050 may comprise a ventilator or other means for the supply of respiratory gases within the required pressure and/or flow range.

The gasses source 1050 is fluidly coupled to an inlet 1034 of a humidifier to supply gases to the humidifier. The humidification chamber 1031 may include a heat source at or adjacent the base of the chamber, for example, the chamber may commonly include a heat conductive base (such as an aluminium base) that sits atop or otherwise contacts a heater plate 1033 of the humidifier 1030. The humidifier 30 may include a control system and/or sensors for controlling the humidifier, for example by controlling the heat plate.

The control system 1036 may include a microprocessor-based controller executing computer with software commands stored in associated memory. The control system may receive input from a user input means such as via buttons or a dial through which a user of the device may, for example, set a predetermined required value (pre-set value) of humidity or temperature of the gases supplied to patient 1010. In response to the user input and/or other inputs from sensors, for example, sensors measuring gas flow or temperature, the controller may determine when (or to what level) to energise the heater plate 1033 to heat the volume of water within humidification chamber 1031. As the water within humidification chamber 1031 is heated, water vapour fills the volume of the chamber above the surface of the water and exits the humidification chamber outlet 1035 with the flow of gases (for example air) flowing from the gases source 1050 through the humidification chamber.

The respiratory conduit 1040 is connected to the outlet 1035 of the humidification chamber 1031, to receive the humidified gases and to deliver these to the patient interface assembly 1020. A conduit heating element 1041 may be provided within the respiratory conduit 1040 to help reduce, prevent, or minimise condensation of the humidified gases within the conduit.

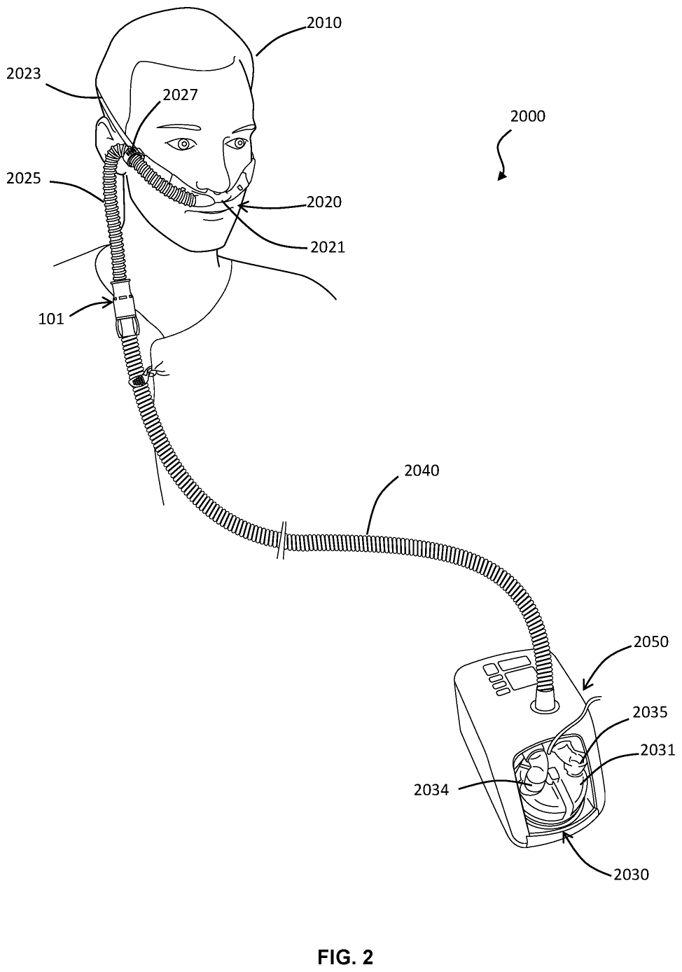

FIG. 2 illustrates an alternative respiratory therapy system 2000 in which the gases source 2050 and humidifier 2030 are both provided within a common housing. Reference numerals are used to highlight analogous components compared to the system 1000 of FIG. 1, with the addition of 1000.

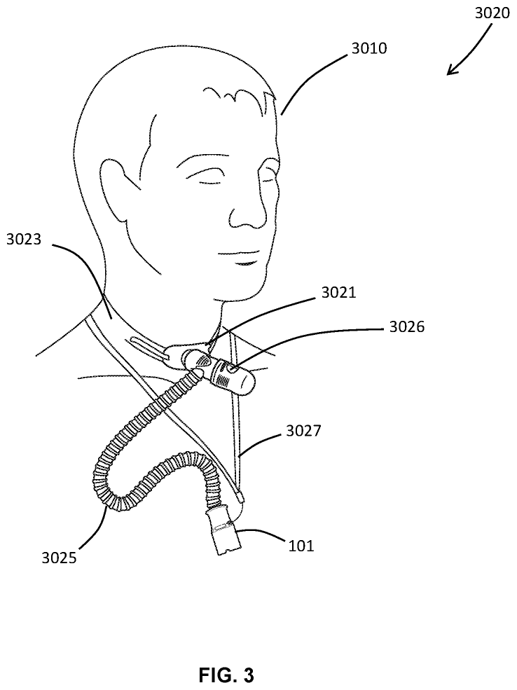

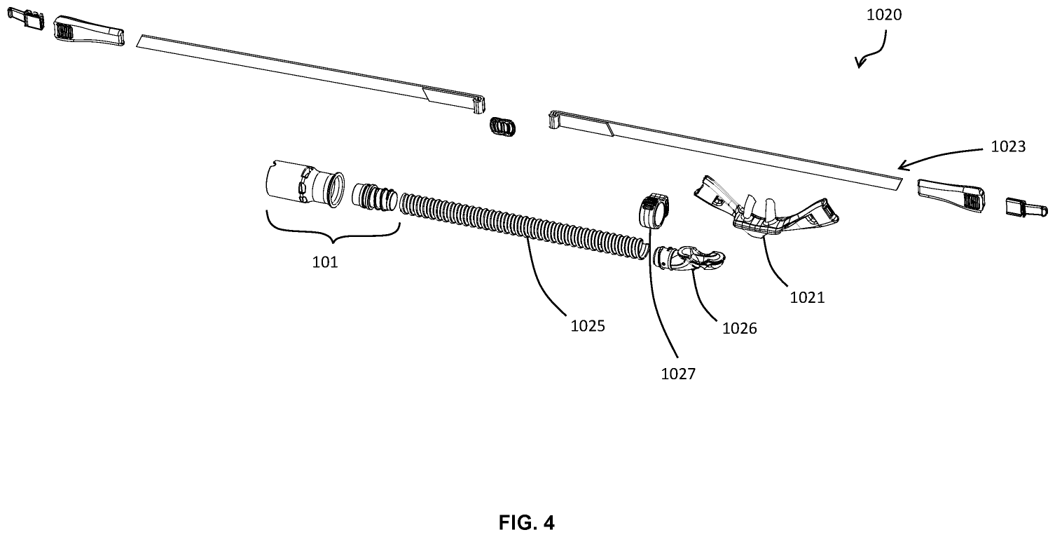

Referring also to FIGS. 3 and 4, the patient interface assemblies 1020, 2020 each comprise a patient interface body 1021, 2021 for delivering gases to the patient 1010, 2010, a securement mechanism 1023, 2023 such as headgear or a strap for securing the patient interface in position relative to the patient 1010, 2010, and a patient interface conduit 1025, 2025 for conveying the gases from the respiratory conduit 1040, 2040 to the patient interface body 1021, 2021.

The patient interface conduit 1025, 2025 is connected at a first end to the patient interface via one or more coupling components 1026 and may optionally be further secured in position relative to the patient by a retention means such as a clip 1027, 2027 (illustrated in FIG. 2). A second end of the patient interface conduit 1025, 2025 can be coupled to the respiratory conduit 1040, 2040 by a connector 101, described in more detail herein.

In some embodiments such as those illustrated in FIGS. 1, 2, and 4, the patient interface assembly 1020, 2020 includes a nasal cannula 1021, 2021. The nasal cannula 1021, 2021 may comprise one or more nasal delivery elements for conveyance of gases into the nostrils of a patient. A nasal delivery element may comprise a prong or a nasal pillow to be inserted into a nostril of a patient to deliver respiratory gases. A nasal delivery element may also comprise an orifice without a nasal prong or pillow, where the nasal delivery element is not inserted into a nostril of a patient to deliver respiratory gases in use. A nasal delivery element may be configured to seal, partially occlude, or not seal with a nostril. A prong may refer to a nasal delivery element configured to partially occlude or not seal with a nostril.

The cannula may be a high flow nasal cannula configured for flow-controlled respiratory therapy. Nasal high flow (NHF) typically uses a non-sealing nasal cannula to deliver a relatively high-volume flow to the nostrils of a patient.

In some examples, the nasal cannula may comprise asymmetrical nasal delivery elements. Alternatively, the nasal cannula may comprise symmetrical nasal delivery elements.

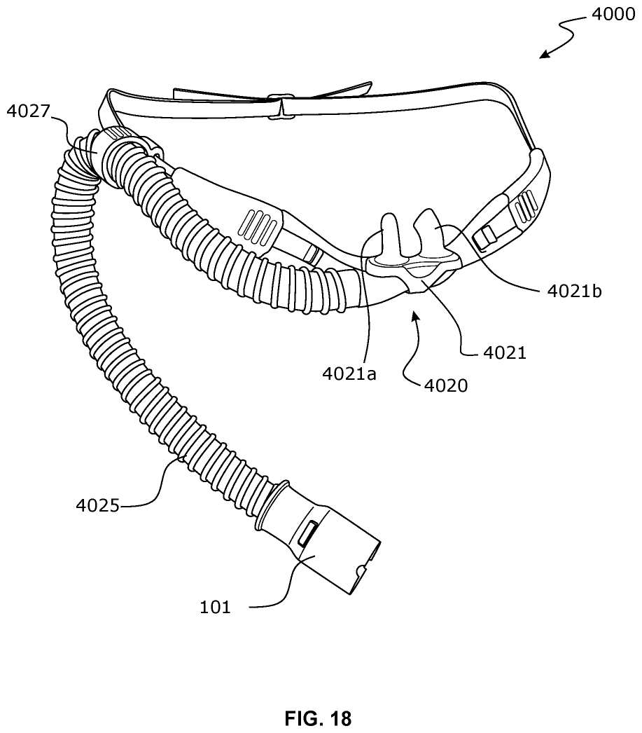

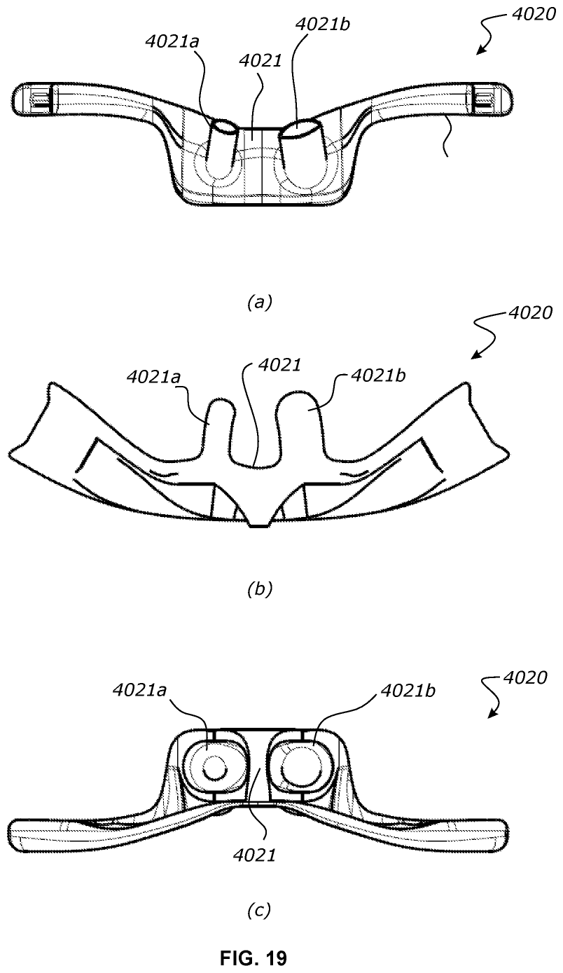

FIGS. 18 and 19 illustrate an example embodiment nasal cannula 4020 having asymmetric nasal delivery elements 4021a, 4021b, for use with the connector/system described herein. Asymmetrical nasal delivery elements may differ in size such as internal and/or external transverse dimensions or diameters, and/or internal and/or external cross-sectional areas. The external cross-sectional area is the cross-sectional area bounded by the outer wall of the nasal delivery element.

A nasal cannula which comprises asymmetrical nasal delivery elements such as the one shown in FIGS. 18 and 19 may cause an asymmetrical flow at a patient. For example, asymmetrical nasal delivery elements may cause an asymmetrical flow at the nostrils of a patient. Asymmetrical flow as described herein refers to a flow that differs within the interface or within the nose or within the interface and the nose. In this way, a different flow may be delivered by each nasal delivery element, or the flow may differ between inspiration and expiration, or the delivered flow may be a combination of the above. Delivery of an asymmetrical flow may improve clearance of dead space in the upper airways, decrease peak expiratory pressure, increase safety of the therapy particularly for children and infants, and reduce resistance to flow in the interface.

Example embodiments of nasal cannulas comprising asymmetrical nasal delivery elements are described in United States Patent Application Publication No. 2016/0158476, the entirety of which is incorporated herein by reference. Further example embodiment nasal cannulas comprising a first prong and a second prong which are asymmetrical to each other are described in WO 2022/229909, the entirety of which is incorporated herein by reference.

In other embodiments, the patient interface coupled to the respiratory conduit 1025, 2025 may include a mask. For example, a nasal mask, a sub-nasal mask, an oro-nasal mask, an oral mask or a full-face mask. The patient interface assembly may comprise a sealing interface for use with CPAP, NIV, and pressure-controlled therapies rather than flow-controlled therapies. Some example interfaces and systems are described in WO 2021/060992, the entirety of which is incorporated herein by reference.

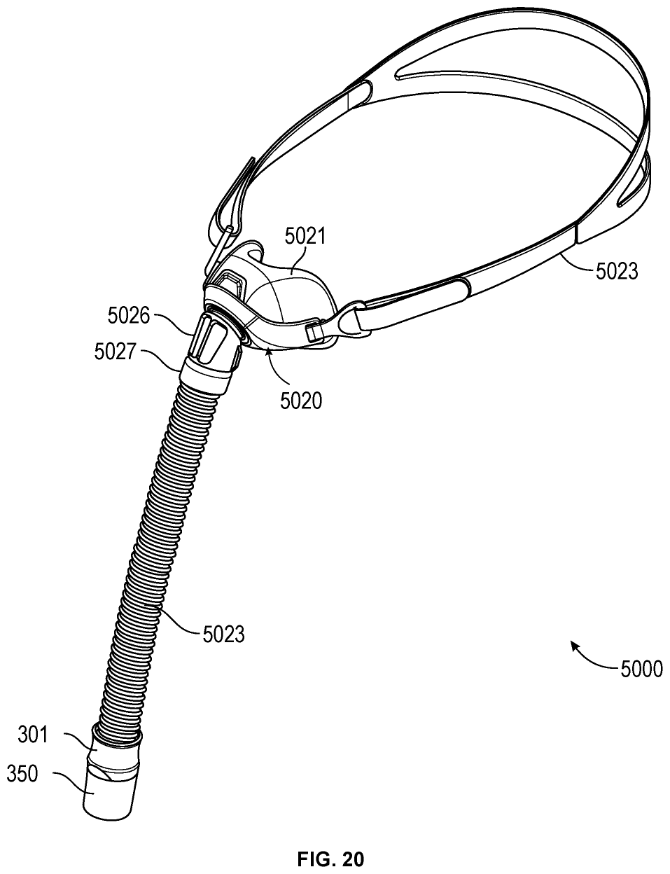

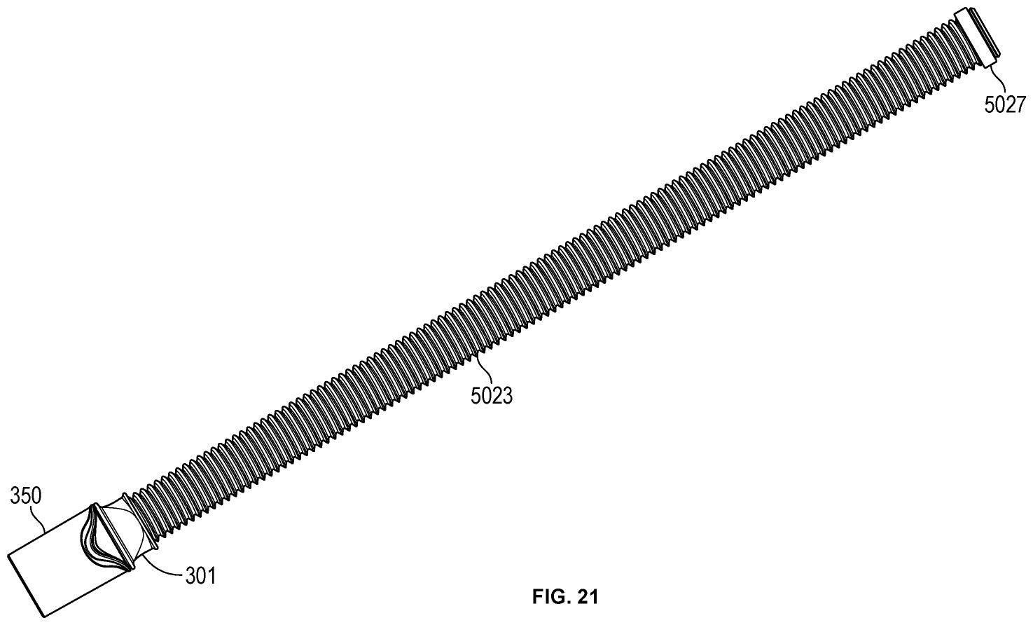

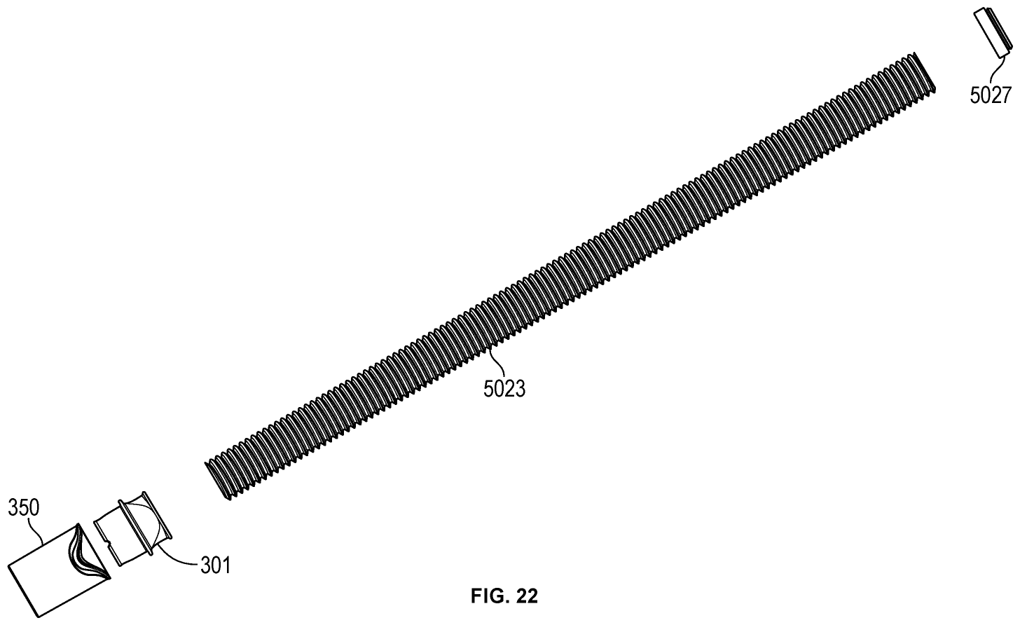

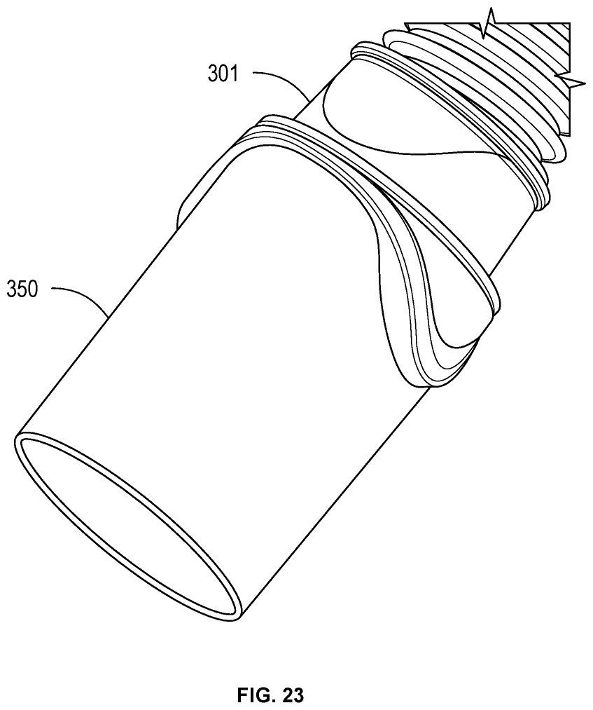

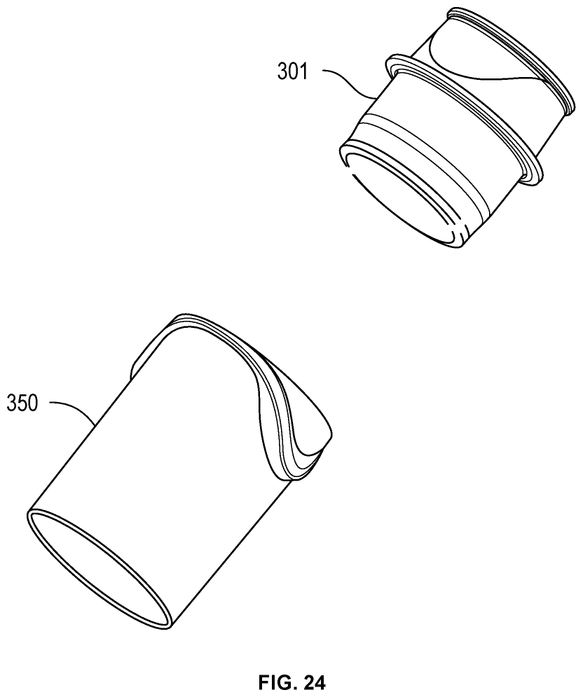

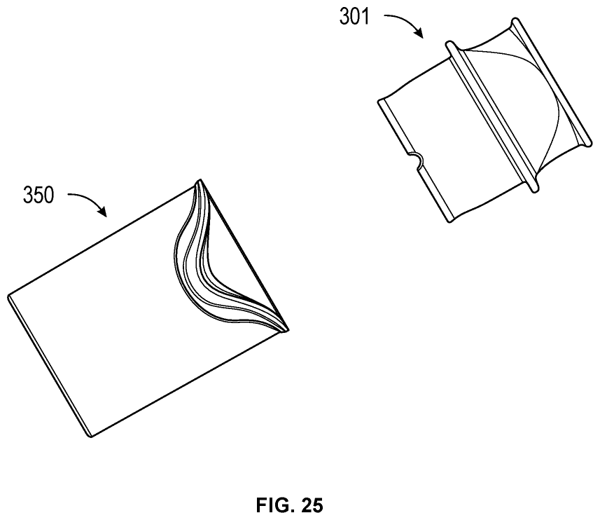

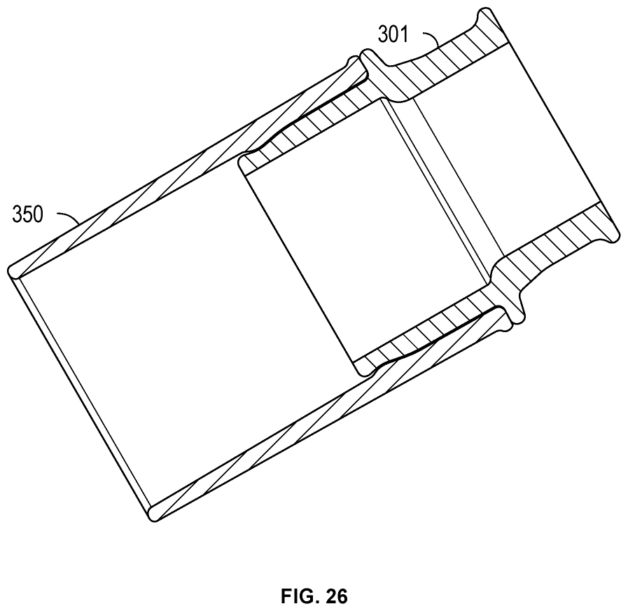

FIGS. 20 to 26 illustrate a system 5000 having a tube assembly for connection to a patient interface 5020 via a coupling component 5026 of the patient interface. A connector 5207 may be provided to couple the patient interface to the tube assembly. The connector 5207 may be configured to couple the coupling component 5026 and the tube assembly. The tube assembly or the coupling component 5026 of the patient interface may comprise the connector 207. The tube assembly may include a length of respiratory conduit 5023 and a connector 301 forming a swivelling connection with a swivel connector 350. The connector 301 is shown schematically in a form that is overmoulded onto the conduit 5023. However, the connector 301 may be as described above in relation to the connectors 101 and 201, that threads into engagement with the conduit 5023. Similarly, the connector 5207 may be as described above in relation to the connectors 101 and 201.

As a further alternative, the patient interface assembly may comprise a tracheostomy tube tracheal adaptor 3021 for connection to a tracheal tube, as illustrated in the patient interface assembly 3020 of FIG. 3. In this embodiment, the tracheostomy tube 3021 is secured in place by a neck strap 3023, and the second end of the patient interface conduit 3025 is tethered to the patient by a lanyard at the connector 101 (shown in part), to prevent the patient interface conduit 3025 pulling down on the patient interface 3021.

In some embodiments, the respiratory therapy system 1 may further comprise an expiratory conduit or exhalation pathway, a means for the supply of nebulised medicament, respiratory filters for removal of contaminants from respiratory gases, and/or any number of ancillary components and connections.

### Patient Interface Conduit

The patient interface conduit 25, 1025, 2025, 3025 will now be described primarily with relation to the patient interface assembly 1020 illustrated in FIGS. 1 and 4, although it will be understood that this description also relates to the patient interface conduits 25, 2025, 3025 in other embodiments such as those in FIGS. 2 and 3 and to patient interface conduits in alternative embodiment systems which may have alternative patient interface assemblies.

Referring to FIGS. 6 and 7, the patient interface conduit 1025 may comprise a first elongate member 1081, 2081 and a second elongate member 1082, 2082 spirally wound together to create a hollow member defining a lumen for the flow of respiratory gases.

The first elongate member 1081, 2081 may be an elongate bead of any suitable cross section. The bead typically has a solid cross-section with a rounded shape, for example, among other shapes it may be round, oblong, semi-circular.

The second elongate member 1082, 2082 may be a thin wall member. For example, comprising a film or membrane or other flexible thin-wall material. The second elongate member 1082, 2082 may have two substantially parallel side edges. The second elongate member 1082, 2082 has a width between it's side edges that is much larger than the wall thickness of the member.

The first and second elongate members are wound together with the second member 1082, 2082 overlapping itself at its side edges for each wind with the first elongate member 1081, 2081 disposed at this overlapping region. In an example, the first member 1081 may be disposed between successive winds of the second elongate member 1082 as shown in FIG. 6. In another example, successive winds of the second elongate member 2082 may form an overlapping portion where the successive winds of the second elongate member 2082 contact or engage each other, and a part of the overlapping portion may be disposed on an internal and/or external surface of the first member 2081, as shown in FIG. 7. The terms ‘internal’ and ‘external’ are taken in reference to the conduit 1025, where an internal surface refers to a surface facing an interior of the conduit 1025 (for example a surface facing the gas flow path through the lumen of conduit 1025) and an external surface refers to a surface facing an exterior of the conduit 1025 (for example a surface facing an ambient environment). The first and second elongate members may be bonded to one another. Successive winds of the first elongate member 1081, 2081 may be bonded to each other. Successive winds of the second elongate member 1082, 2082 may be bonded to each other.

In the embodiment of FIG. 7, the two overlapping edges of the second elongate member 2082 are bonded to the first elongate member 2081 and optionally to each other to form the hollow conduit of the patient interface conduit.

The first elongate member 1081, 2081 creates a helical ridge on an external surface of the conduit. The helical ridge may have a constant bead pitch, for example, between about 2.6 mm and about 5 mm. In the embodiment shown, the helical bead 1081, 2081 has a pitch of about 4.5 mm, a thickness/height of about 1 mm, and a width of about 2 mm. In alternative embodiments, the bead may have a height of between about 0.5 mm and about 3 mm and a width of between about 1 mm and about 2 mm.

The internal surface of the conduit may be substantially smooth as illustrated in the embodiment of FIG. 6 or may also have an undulating surface as illustrated in FIG. 7. In an embodiment, the resulting conduit has an inner diameter of about 12 mm and an outer diameter of about 14 mm. However, in alternative embodiments, the inner diameter may be any diameter between about 10 mm and about 14 mm; and the outer diameter may be any diameter between about 12 mm and about 16 mm.

The first elongate member 1081 and the second elongate member 1082 may be formed from any suitable material, most commonly a polymer. The first elongate member 1081 and the second elongate member 1082 may be formed from the same materials or polymers, or different materials or polymers. The first elongate member 1081 may be more rigid than the second elongate member 1082.

The second elongate member 1082, 2082 may comprise a breathable film. As used herein, “breathable” is used to describe a material that allows the passage of water molecules through a monolithic wall of the material via the solution-diffusion mechanism, without allowing the bulk passage of liquid water or bulk flow of respiratory gases all the way through the wall. It will be appreciated by one of skill in the art that the water molecules in the wall are molecularly dispersed in the media, and are therefore without a state (solid, liquid, or gas), although they are sometimes referred to in the art as vapour (e.g., the rate of transfer is often referred to as a water vapour transmission rate or the like). It should further be appreciated that a monolithic wall does not contain open channels or through holes from one major surface to another, such that viruses could be carried through such channels or holes alongside air or liquid water drops via the pore flow mechanism. It should yet further be appreciated that, like all polymers, some small molecule transport of respiratory gases (such as oxygen, carbon dioxide or nitrogen) may occur in trace or de minimis amounts (i.e., not “bulk” flow), which, for a breathable material as defined herein, would typically be at a rate at least an order of magnitude lower than that for water molecules. Furthermore, of particular relevance for breathing gases being delivered to or from a patient, such small molecule transport of respiratory gases would be of an amount less than that allowed for compliance with the relevant standards, for example, in the leakage test of ISO 5367:2014 at Section 5.4 tested via the method set out in Annex E, which is hereby incorporated by reference in its entirety.

The use of a breathable material within the patient interface conduit 25, 1025, 2025, 3025 may help to reduce or prevent condensation of the humidified gases within the conduit. Such condensation is due to the temperature of the walls of the patient interface conduit being close to the ambient temperature, (being the temperature of the surrounding atmosphere) which is usually lower than the temperature of the humidified gases within the conduit.

During the provision of respiratory therapy or respiratory support, properties of the patient interface conduit may change owing to environmental conditions relating to temperature, humidity, or otherwise. The patient interface conduit may be at a temperature of between 25° C. and 55° C., more preferably at 37° C. The flow of gases may have an absolute humidity of greater than 12 mg/L, greater than 33 mg/L, more preferably 44 mg/L. The flow of gases may have a flow rate of between 2 L/min and 60 L/min, such as above 20 L/min. The flow of gases may have a relative humidity of up to 100%. The properties of the patient interface conduit which change may relate to stiffness, flexibility, strength, residual stress, and/or stress relaxation.

In some embodiments, the patient interface conduit 25, 1025, 2025, 3025 may include a heating element, for heating the gasses flowing along the conduit.

### Connector

A connector 101, described in more detail below, is provided to couple the patient interface assembly 1020, 2020, 3020 to the rest of the respiratory system 1000, 2000, 3000. In the embodiments described herein, the connector 101 is utilised to connect the respiratory conduit 1040, 2040 to the patient interface conduit 1025, 2025, 3025. However, the connector 101 may be used for coupling other components and/or conduits together in other systems or applications.

The connector 101 may be located elsewhere in the respiratory system 1000, 2000, 3000 and is not limited to use in the positions shown in the exemplary systems of FIGS. 1 to 3. For example, in some systems, the connector 101 may be located more proximal to the patient interface body 1021, 2021 to facilitate coupling between the patient interface conduit 1025, 2025 and the patient interface body 1021, 2021. For example, the coupling component 1026 of the patient interface 1020 may comprise the connector 101 or a part thereof (for example, the first part 103 of the connector or the protrusion or protrusions 125). In some examples, the connector 101 may be located on an expiratory conduit. In some examples, the connector may be located on an inlet portion and/or outlet portion of a respiratory filter.

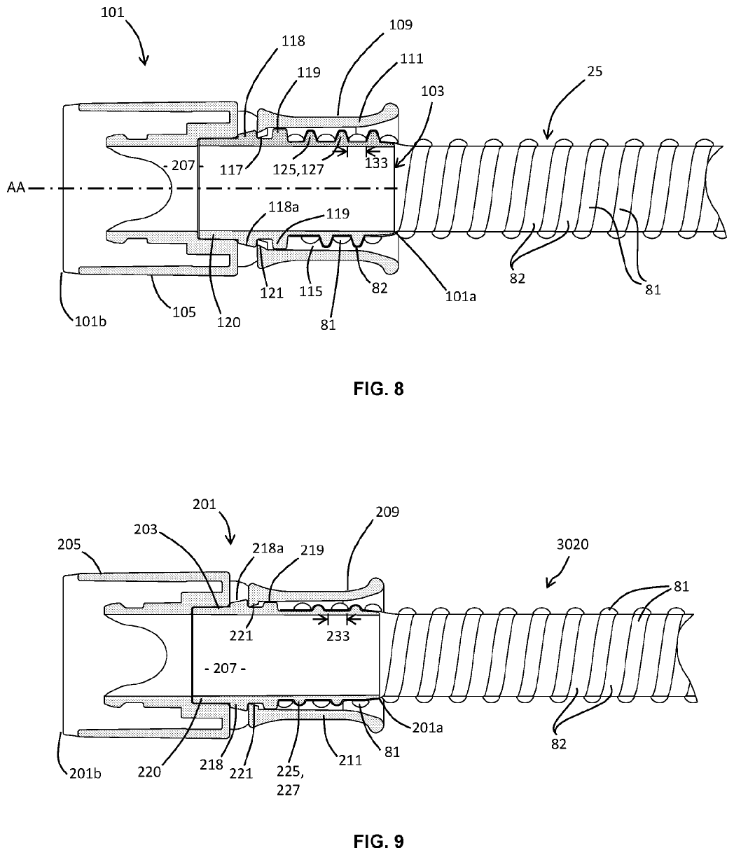

Referring to FIG. 8, the connector 101 has a first part 103 and a second part 105. The first part 103 forms a first end 101a of the connector 101 and the second part 105 forms a second end 101b of the connector 101. Together, the first and second parts 103, 105 define an internal lumen 107 for the passage of gas through the connector 107, for example, gasses received from a respiratory conduit 1040. The first and second parts 103, 105, are preferably coaxial with the internal lumen 107 being generally cylindrical.

In some embodiments there are no abrupt changes along the wall of the internal lumen 107, for example walls of the internal lumen 107 may be substantially contiguous between the first and second parts 103, 105 and/or the internal surface defining the lumen 107 may be smooth. The diameter of the lumen 107 may be constant along the length of the lumen or may vary along the length.

The first part 103 of the connector 101 is configured to engage an end portion of the patient interface respiratory conduit 25 (or another conduit).

The second part 105 of the connector 101 is configured to couple to a complementary connector or component. This coupling may be a removable coupling or a permanent coupling. In an example, the second part 105 may be integral with another component. The complementary connector may be provided at a terminal end of the respiratory conduit 1040 or on another conduit or component to thereby enable coupling of said conduit or component to the patient interface respiratory conduit 25. The engagement between the second part 105 of the connector 101 and the complementary connector may be any suitable connection, for example, a snap-fit connection, a clamping connection, a friction connection, or a threaded connection.

The internal lumen 107 in the connector 101 creates a gases path between the respiratory conduit 1040 and the patient interface respiratory conduit 25, such that the gases can be delivered to the patient interface 1020.

The connector 101 further includes a retention element 109 that is generally external to the first part 103 of the connector 101. The retention element 109 defines an outer wall 111 positioned outwards from an engagement region 104 of the connector first part 103 such that the first part 103 at least partly internal with respect to the retention element 109. The engagement region 104 of the connector first part 103 forms an inner wall 113 of the connector, spaced inwards from the outer wall 111. The inner and outer walls 111, 113 define a cavity 115 therebetween for receiving an end portion of the wall of the patient interface respiratory conduit 25 (or other conduit).

In the embodiments shown, the retention element 109 has the general form of a hollow cylinder positioned co-axially with the first and second connector parts 103, 105. The inner and outer diameters of the retention element 109 being greater than the maximum diameter of the first connector part 103 in the engagement region 104.

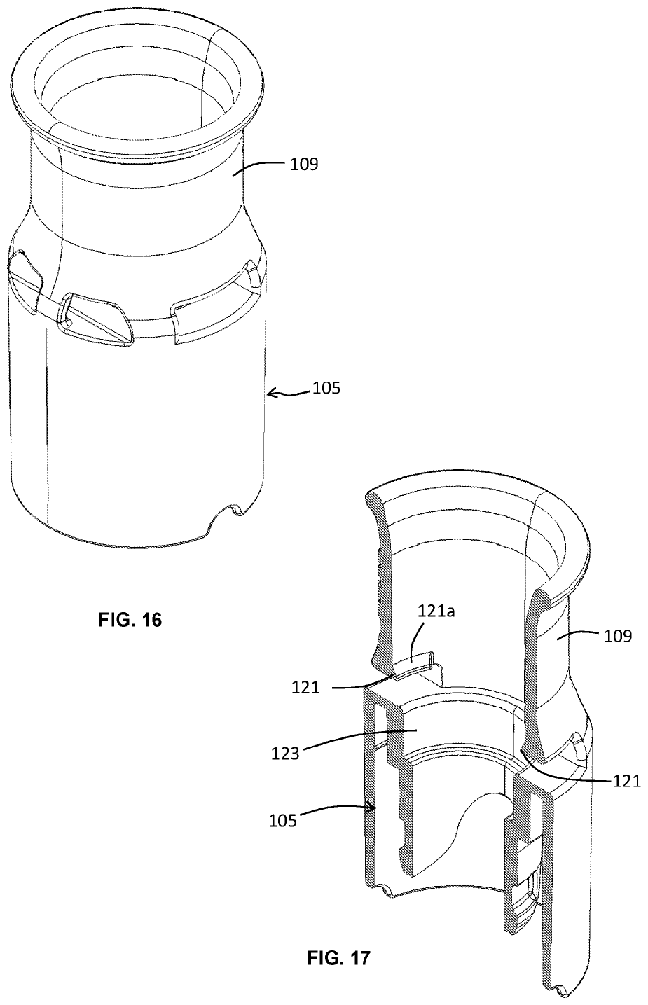

The first and second parts 103, 105 may be separate components or they may be integrally formed. The retention element 109 may be integral with the first connector part 103 or with the second connector part 105, or it may be a separate component that is connectable to the first and/or second connector part(s). In the embodiment shown, the first and second connector parts 103, 105 are separate components, with the retention element 109 being integral with the first connector part 103 (see, in particular, FIGS. 16 and 17).

The connector first part 103 may be rotatable relative to the second part 105. In the exemplary embodiments, the first part 103 is rotatably connected to the second connector part 105 such that the first and second parts 103, 105 are free to rotate relative to each other about the central axis AA of the connector and internal lumen. This rotation may decrease the likelihood of the patient interface conduit becoming tangled or twisted in response to movement of a patient or otherwise.

In the embodiment shown, to facilitate rotational coupling of the first connector part 103 to the second part, the first connector part 103 comprises an annular groove 117 created by two spaced annular projections 118, 119. A plurality of inward projections or detents 121 are provided on the retention element 109, shaped and positioned to engage the annular groove 117. The detents 121 and/or one or both or the annular projections the may be shaped to facilitate a snap fit. For example, on the embodiment, a face 121a of the detent and a surface 118a of a first one of the annular projections are angled such that when the two components 109, 103 are pushed together in an axial direction, the angled surfaces 118a, 121a slide against each other encouraging the detents to flex outwards such that the detent can move over the first annular projection before ‘snapping’ into the recess provided by the annular groove. In the engaged position the contacting or facing surfaces of the first annular projection and the detent are parallel and orthogonal to the axial direction to prevent disconnection of the two components.

Many alternative configurations are envisaged for rotatably coupling the first and second connector parts 103, 105. For example, rather than being provided on the first connector part 103, a groove or shoulder may be provided on the second connector part 105 or on the retention member 109, and one or more complementary outwardly-projecting protrusions may be provided on the first part 103. Rather than discrete protrusions or detents, an annular protrusion may be provided.

The rotational coupling between the first and second components 103, 105 is preferably one that ensures a sufficient pneumatic seal between is formed between the first and second parts to prevent or minimise leakage of gasses at the connector 101. In some embodiments a sealing component such as an o-ring may be fitted between the first and second connector parts 103, 105. However, sealing components may impact on the freedom of the rotatable connection. Alternatively, the clearance between the first and second connector parts 103, 105 may be sufficiently tight such that at operating pressures any leakage between the internal and external components is negligible or insignificant during use, the clearance still being sufficient to allow for relative rotation between the parts.

The length of the bearing surface 123 between the first and second parts 103, 105 may also be selected to reduce leakage, with a longer length of the bearing surface generally reducing leakage. In the exemplary embodiments, the first connector part 103 comprises a boss 120 that is received in a complementary recess in the second connector part and which bears against the second connector part during rotation. The length of this boss may be selected to reduce or minimise leakage between the connector parts.

The connector 100 may have any one or more of the features described in relation to the connector of U.S. patent application Ser. No. 14/861,266 and or U.S. patent application Ser. No. 15/756,953. The contents of which are incorporated herein in their entirety by way of reference.

### Engagement Region of Connector First Part

The engagement region 104 of the first connector part 103 is configured to engage with the wall of the patient interface conduit 25 to secure the connector to the conduit 25. The engagement region 104 includes one or more protrusions 125 for engagement with the conduit wall.

In the embodiments shown, the protrusion or protrusions 125 lie along a substantially helical path, for example, forming a thread so that the first part 103 of the connector can be wound into engagement with the conduit. Successive winds of the thread define a helical groove for receiving the bead of the conduit. The helical path is shaped to correspond to the conduit 25 that will be coupled to the connector first part. For example, the helical path will generally have a pitch that is substantially the same as the pitch of a helical feature, for example a helical bead, on the respiratory conduit.

The lobes 127 may be shaped to facilitate one or more of ease of assembly with the conduit 25, resistance to removal of the conduit 25 by unwinding, and/or shaped to reduce stress concentrations induced in the attached conduit. The height of each lobe varies along the length of the lobe.

Each lobe 127 comprises a leading portion 127a at a leading end of the lobe (with respect to the helical path) and a trailing portion 128b at a trailing end of the lobe. During assembly of the connector first part 103 with the respiratory conduit 25, the leading portion 127a of each lobe 127 engages the wall of the conduit 25 before the respective trailing portion 127b.

In some embodiments, the trailing portion 127b of each lobe 127 has a generally steeper gradient with respect to a surface of the engagement region 104 than the gradient of the leading portion 127a. The length, measured along the helical path, of the leading portion 127a of the lobe may therefore be longer than the length of the respective trailing portion 127b. Referring to FIG. 15, in the embodiment shown, the leading portion 127a of each lobe 127 extends along an arc length La, the trailing portion 127b of each lobe 127 extends along an arc length La that is about one third the length of La. In alternative embodiments other ratios between the lengths of the leading and trailing portions are envisaged, for example the leading portion 127a of each lobe 127 may extend along an arc length that is between about 1 and about 5 times the length of the trailing portion 127b, preferably between about 2 and about 4 times the length of the trailing portion 127b. In the exemplary embodiment shown, the leading portion 127a of each lobe 127 may extend along an arc length that is about 3.2 times the length of the trailing portion 127b.

The height of each lobe varies between their respective leading and trailing ends. The maximum height of each lobe occurs at a region or point mp that is intermediate the respective leading and trailing ends. In the embodiments described herein, the maximum height occurs at a point mp that is closer to the trailing end of the lobe 127 than to the leading end of the respective lobe 127. In some embodiments, the maximum lobe height occurs along a length of the lobe 127 intermediate the leading and trailing portions 127a, 127b. In such embodiments, each lobe 127 generally has a lower height proximal the leading end than the height proximal the trailing end. The gradient of the lobe leading and trailing portions 127a, 127b are non-linear and vary along the lobe, typically being steepest near the respective end of the lobe and flattest near the maximum height region or point mp of the lobe 127.

In the embodiment shown, the lobes 127 have a shape in which the leading and trailing portions are continuous, joining at a point or region of maximum height, such that the profile of the lobe 127 has convex curvature along the length of the lobe. However, other lobe profiles are envisaged. For example, in some embodiments, each lobe may have a portion intermediate the leading and trailing portions having a local minima and/or including a convex portion.

The maximum height H of each lobe 127 of the engagement region 104 may be substantially the same for each lobe or may vary between lobes. In one embodiment, the maximum height H of the lobe closest the first end 101a of the connector is shorter than the maximum height H of the lobe farthest from the first end 101a of the connector. The height of successive lobes may increase gradually from the lobe closest the first end 101a of the connector to the lobe farthest from the first end 101a, or the heights may only increase for the first few lobes before and remaining constant for the remaining lobes.

The lobes 127 may be formed by a single shaped helical protrusion or may be formed by a series of discrete protrusions on the first connector part 103. The leading end of each lobe 127 may be spaced from or may contact, or be contiguous with, the trailing end of the previous lobe. In some embodiments, other projections or features may be present between lobes.

The overall length of each lobe 127, measured along the helical path, may be the same for each lobe 127 in the engagement region 104, or different lobes may be of different lengths. In the embodiments shown, the length of each lobe 127 is the same, other than a first engagement lobe 128 at the first end of the connector, the leading portion of which has a shorter length and a steeper gradient than the other lobes 127.

The number of lobes provided and/or the number of lobes for each coil of the helical path may vary between embodiments. In the embodiments shown, the lobe extends along a 90 degree arc θ, and are substantially contiguous such that there are four lobes 127 for each coil of the helical path.

In alternative embodiments, the lobes 127 may be shorter or longer with respect to the connector body, and/or the lobes may be spaced apart. As a non-limiting example, in other embodiments each lobe may extend along an arc having an angle θ between about 30 degrees and about 360 degrees, preferably between about 45 and 120 degrees. For example, in other embodiments there may be between one and eight lobes for each coil of the helical path, however alternatively there may be less than one or more than eight. In some embodiments, the number of lobes per coil may not be a whole number, for example, there may be 2.5 or 5.3 lobes per rotation.

The side edges of the lobes 127 are rounded or otherwise shaped such that there are no sharp corners on the lobe. This is intended to reduce stress concentrations in the respiratory conduit when engaged. In the embodiment shown, the outer edges 131 of the lobes 127 are filleted. The radius of the fillets on the lobe edges generally will depend on the dimensions of the lobe. The two edges may have fillets that are the same size, or different, and/or the fillet radius may vary along the length of the lobe. For example, the radii of the fillets may be less at the base of the lobes and greater near the maximum height point, and/or the fillets at the leading portion of a lobe may differ from those for the trailing portion.

The width of the base of the, or each, projection is preferably selected so that the spacing 133 between successive coils of the projection(s) is equal to or greater than the cross-sectional width of the conduit bead. In the embodiment illustrated in FIG. 8, the width of the helical groove at the base of each lobe 127 is about the same as the cross-sectional width of the conduit bead such that the bead sits snugly in the helical groove.

In an alternative embodiment shown in FIG. 9, the width of the helical groove at the base of the lobes 227 is greater than the cross-sectional width of the conduit bead 81 such that the bead may be able to move a short distance in the connector axial direction, within the helical groove, upon application of an axial force. In this embodiment, the maximum height of the lobes 227 is less than for the lobes 127 in the previous embodiment 101 such that the inner surface of the retention element 209 is positioned closer to the conduit beads 81. In this specific example, the height of the lobes 227 is less than the bead thickness, such that the beads are closer to the retention element inner surface than the lobes. This embodiment with shallower lobes may result in less tension in the wall of the conduit 25.

In the embodiment of FIG. 9, like reference numbers are used to indicate like features. That is, unless otherwise described, like reference numbers are used for like or similar features but with the addition of a of 100, for example 101, 201.

Engagement with Conduit

To engage the connector 101 with the patient conduit 25, the first part 103 of the connector is wound into engagement with the conduit. The first part 103 of the connector may be wound into engagement with the conduit before being assembled with the other connector component(s). The shallower leading portions 127a of the lobes 127 reduces the torque required to wind the first connector part 103 into engagement with the conduit for easier assembly.

As the first part 103 of the connector 101 is wound into engagement with the conduit 25, the thread formed by the lobes 127 is positioned between successive winds of the conduit bead 81, in contact with the wall/film of the conduit 25. The bead of the patient interface conduit guides the thread. The leading portion 127a of each lobe 127 engages the conduit wall before the respective trailing portion 127b. The lobes may be shaped to have an interference fit with the wall of the conduit along a portion of the lobe such that the wall is deflected and stretched over the lobe, introducing tension.

The winding is continued until a length of the conduit 25 is seated on the first connector part, for example along substantially all of the engagement region. The first connector part may include a stop to arrest winding of the part along the conduit. In the embodiment shown, a side of one of the annular projections 119 acts as a stop to define when the conduit is fully engaged. When the conduit 25 is seated on the first connector part 103, the bead 81 is seated on the first connector part between the successive coils of the thread 125 formed by the lobes 127. The film 82 of the conduit stretches over the thread 125.

Due to the variation in height along each lobe 127, when the connector is engaged with the conduit, the tension in the wall of the conduit 25 varies along the length of each lobes and along the helical path. Maximum tension may typically occur in the film of the conduit wall, proximal the maximum height points of each lobe 127. Contact of each lobe may be along a portion or along substantially all of the lobe.

The lobes 127 are shaped to minimise stress concentration factors induced in the film adjacent the lobes and/or to minimise area with increased stress concentrations, and/or to reduce the area of the film that is under maximum tension. This has the effect of reducing the stress that the wall of the conduit 25 experiences during engagement and the stress that the wall of the conduit 25 experiences under to an applied load, thereby reducing the likelihood of a tear in the conduit 25 during assembly and increasing the force that the assembled conduit can tolerate before tearing.

The shape of the lobes 127 may be such that the torque required to unwind the conduit from the connector is greater than the torque to wind the conduit on to the connector, thereby providing some resistance to inadvertent unwinding of the connection.

To assemble the connector in the exemplary embodiments, the first part 103 of the connector 101 is first engaged with the interface conduit 25, for example by winding the components together as described above. This forms a pneumatic seal between the interface conduit and the first part of the connector to allow gasses flow from the first connector part 103 into the interface conduit without non-negligible gas leakage. In a second step, the boss 120 end of the first connector part 103 is then pushed into engagement with the second connector part 105 (inclusive of the retention element 109) causing the engagement of the annular groove 117 on the first part with the detents 121 on the retention element.

Assembly of the connector 101 in the exemplary embodiments avoids the use of adhesives or overmoulding for connection with a conduit or with other components. In some alternative examples, the assembly of the connector may comprise the use of adhesives or overmoulding to improve a connection.

### Spacing of Retention Element

The connector outer wall 111 defined by the retention element 109 is spaced outwards from the outer surface of the engagement region 104 of the connector first part 103. This forms a generally annular cavity therebetween. The spacing between these components is selected to inhibit or reduce the likelihood of the interface conduit 25 being disconnected from the connector upon experiencing an axial force, for example when a tensile force or another force having a tensile axial component is applied to the interface conduit 25. This is achieved by inhibiting the portions of the conduit wall having maximum thickness, such as the wall bead described above, from being pulled over the one or more protrusions on the first connector part.

The smallest spacing between an inner surface of the retention element 109 and the outer surface of the connector first part in the engagement region is less than the maximum wall thickness of the conduit 25 such that the conduit cannot easily be pulled through the annular cavity at maximum thickness point.

The smallest spacing between the inner surface of the retention element 109 and the outer surface of the connector first part 103 may occur at a single point or region, but more preferably occurs at a plurality of points or regions. In the exemplary embodiments, the closest distance between the retention element 109 and the engagement region 104 of the first connector part occurs at the maximum height point of the lobes 127.

The smallest spacing between an inner surface of the retention element 109 and the outer surface of the connector first part 109 in the engagement region 104 may depend on the properties of the conduit wall, for example the stiffness of the bead, and/or properties of the retention component such as its flexibility. The spacing should be 1% less than the maximum wall thickness of the conduit wall, or smaller. The spacing should also be larger than the minimum wall thickness of the conduit 25 to allow the retention element to be positioned over the engaged conduit 25.

In the embodiments 101, 201 illustrated, the smallest spacing is about 55% less than the maximum wall thickness of the conduit wall. For example, for a conduit with a bead thickness of 1 mm and a film thickness of about 50 μm, the internal surface of the retention element is spaced about 0.45 mm from the maximum height points mp of the projections.

The inner surface of the retention element 109 may have a constant diameter along a major portion of the retention element, or it may vary. In particular, the internal diameter of the retention element may increase, for example by the wall of the element flaring outwards, at the end proximal the first end 101a of the connector 101. This may assist with assembly by guiding the first connector part 103 into the interior of the retention element 109.

### Use of the Connector and Failure Mode

Once the connector 101, 201 is engaged with the patient interface conduit 25 and assembled, it may be difficult for the patient interface conduit 25 to be removed from the connector, particularly for embodiments where the first connector part 103 is rotatable relative to the second connector part 105. Therefore, the connector inhibits accidental decoupling of the interface conduit 25 from the connector due to axial rotation of the conduit or connector.

The retention element 109 prevents the patient interface conduit 25 being pulled intact from the connector 101 under tensile axial loading. In embodiments with such a retention component, the first failure mode is most typically tearing of the wall of the patient interface conduit 25 when the axial loading thereof is above a threshold force. In some embodiments, the strength of the coupling between the first and second connector parts is sufficiently strong that the first and second connector parts 103, 105 will remain connected under axial loading that is at least as great as the threshold force causing tearing of the conduit wall.

The failure of the connection between the connector and patient interface conduit under tensile axial loading becomes dependent on the tensile strength of the conduit wall, for example of the film, rather than the stiffness of components of the connector or the stiffness of the conduit. This may enable the connection to withstand higher tensile loads before failure compared to prior art connectors.

Preferred embodiments of the invention have been described by way of example only and modifications may be made thereto without departing from the scope of the invention.

Alternative embodiments having only some of the features described herein are envisaged, for example, a connector may include an engagement region having an engagement thread formed from a plurality of outwardly projecting lobes, but without a retention element, or with a different retention element spaced as described herein. As a further example, in another embodiment, the connector may include an engagement region that includes a helical engagement projection without lobes or without height variation along the helical projection or that includes another engagement feature in combination with the retention element described herein.

1. A connector for a respiratory support system, comprising:
a first part configured to engage a portion of a respiratory conduit and a second part configured to engage with another component, together, the first and second parts defining an internal lumen for the passage of gas through the first and second parts; and
a retention element defining an outer wall;
wherein the first part of the connector comprises a wall that is spaced inwards from the outer wall, thereby defining a cavity between said wall and the outer wall to receive said portion of the respiratory conduit, the wall of the first part having one or more outwardly-projecting protrusions configured to engage said portion of the respiratory conduit; and
wherein at least one distance between a surface of the protrusion(s) and an inner surface of the outer wall is less than a maximum wall thickness of the portion of the respiratory conduit.

2. A connector as claimed in claim 1, wherein the connector is configured to locate the maximum wall thickness of the portion of the respiratory conduit between the outwardly-projecting protrusion(s).

3. A connector as claimed in claim 1 or 2, wherein the closest distance between the, or each, protrusion and the inner surface of the outer wall is less than a bead size of the conduit.

4. A connector as claimed in any preceding claim, wherein at least one distance between an outer surface of the wall of the first part and an inner surface of the outer wall is greater than the maximum wall thickness of the portion of the respiratory conduit.

5. A connector as claimed in any preceding claim, wherein at least one distance between a surface of the, or each, protrusion and an inner surface of the outer wall is greater than the maximum wall thickness of the portion of the respiratory conduit.

6. A connector as claimed in any preceding claim, wherein the closest distance between the, or each, protrusion and the inner surface of the outer wall is greater than a minimum wall thickness of the respiratory conduit.

7. A connector as claimed in any preceding claim, wherein, when the conduit is connected with the second part, the conduit wall is spaced inwards from the inner surface of the outer wall.

8. A connector as claimed in any preceding claim, wherein the protrusion(s) forms a plurality of lobes aligned along a substantially helical path; and
wherein each lobe comprises a leading portion at a leading end of the lobe and a trailing portion at a trailing end of the lobe, the trailing portion having a steeper gradient with respect to a surface of the first part than the leading portion.

9. A connector as claimed in claim 8, wherein, during assembly of the first part of the connector with the respiratory conduit, the leading portion of each lobe engages the wall of the conduit before the respective trailing portion.

10. A connector as claimed in claim 8 or 9, wherein the lobes are formed by a single helical protrusion

11. A connector as claimed in claim 8 or 9, wherein the lobes are formed by a plurality of discrete protrusions.

12. A connector as claimed in any one of claims 8 to 11, wherein a maximum height of each lobe is at a point closer to the trailing end of said lobe than the leading end of the lobe.

13. A connector as claimed in any one of claims 8 to 12, wherein the leading end of each lobe, the trailing end of each lobe, and an axis of the connector form an angle of about 90 degrees.

14. A connector as claimed in any one of claims 8 to 13, wherein four lobes are provided for each coil of the helical path.

15. A connector as claimed in any one of claims 8 to 14, wherein the helical path has a pitch that is substantially the same as the pitch of a helical feature on the respiratory conduit.

16. A connector as claimed in any preceding claim, wherein the one or more protrusions are configured such that a torque required to wind the conduit onto the connector is less than a torque required to unwind the conduit from the connector.

17. A connector as claimed in any preceding claim, wherein the first part of the connector is rotatably connected to the second part of the connector.

18. A connector as claimed in any preceding claim, wherein a first end of the retention element is engaged with the connector.

19. A connector as claimed in any preceding claim, wherein the retention element comprises a sleeve.

20. A connector as claimed in any preceding claim, wherein the at least one distance between a surface of the protrusion and an inner surface of the outer wall is selected to inhibit or prevent disconnection of the respiratory conduit from the connector due to a generally axial force.

21. A patient interface assembly for a respiratory support system comprising:
a patient interface;
a respiratory conduit connected to the patient interface; and
a connector as claimed in any one of claims 1 to 2019;
wherein the first part of the connector engages an end portion of the respiratory conduit.

22. A patient interface assembly, as claimed in claim 21, wherein a wall of the conduit comprises a flexible film.

23. A patient interface assembly, as claimed in claim 22, wherein the flexible film is breathable.

24. A patient interface assembly, as claimed in any one of claims 21 to 23, wherein a wall of the conduit comprises a helical bead.

25. A connector for a respiratory support system, comprising:
a first part configured to engage a portion of a respiratory conduit, and second part configured to engage with another connector, the first and second parts together defining an internal lumen for the passage of gas through the first and second parts; and
a retention element defining an outer wall;
wherein the first part comprises a wall spaced inwards from the outer wall, forming an inner wall of the connector and thereby defining a cavity between the inner and outer walls to receive said portion of the respiratory conduit, the inner wall having a protrusion configured to engage said portion of the respiratory conduit; and
wherein a distance between a surface of the protrusion and an inner surface of the outer wall is selected to inhibit or prevent disconnection of the respiratory conduit from the connector due to a generally axial force.

26. A connector as claimed in claim 25, wherein the distance between a surface of the protrusion and an inner surface of the outer wall is selected such that, upon application of an axial force to the conduit that is above a threshold force, the wall of the conduit will tear while the second part of the connector remains engaged with the conduit.

27. A connector as claimed in claim 25 or 26, wherein at least one distance between a surface of the protrusion and an inner surface of the outer wall is less than a maximum wall thickness of the respiratory conduit.

28. A connector as claimed in claim 27, wherein the closest distance between the protrusion and the inner surface of the outer well is less than a bead size of the conduit.

29. A connector as claimed in any one of claims 25 to 28, wherein at least one distance between an outer surface of the inner wall and an inner surface of the outer wall is greater than a maximum wall thickness of the respiratory conduit.

30. A connector as claimed in any one of claims 25 to 29, wherein the closest distance between the protrusion and the inner surface of the outer wall is greater than a minimum wall thickness of the respiratory conduit.

31. A connector as claimed in any one of claims 25 to 30, wherein, when the conduit is connected with the connector, the conduit wall is spaced inwards from the inner surface of the outer wall.

32. A connector as claimed in any one of claims 25 to 31, wherein the first part of the connector comprises one or more protrusions that form a plurality of lobes aligned along a substantially helical path; and
wherein each lobe comprises a leading portion at a leading end of the lobe and a trailing portion at a trailing end of the lobe, the trailing portion having a steeper gradient with respect to a surface of the first part than the leading portion.

33. A connector as claimed in claim 32, wherein, during assembly of the first part of the connector with the respiratory conduit, the leading portion of each lobe engages the wall of the conduit before the respective trailing portion.

34. A connector as claimed in claim 32 or 33, wherein the one or more protrusions form a continuous or discontinuous thread.

35. A connector as claimed in any one of claims 32 to 34, wherein the height of each lobe, from the base of the lobe to the top of the lobe, varies along the lobe from the leading portion to the trailing portion.

36. A connector as claimed in any one of claims 32 to 35, wherein the lobes are formed by a single helical protrusion.

37. A connector as claimed in any one of claims 32 to 35, wherein the lobes are formed by a plurality of discrete protrusions.

38. A connector as claimed in any one of claims 32 to 37, wherein a maximum height of each lobe is at a point closer to the trailing end of said lobe than the leading end of the lobe.

39. A connector as claimed in any one of claims 32 to 38, wherein the leading end of each lobe, the trailing end of each lobe, and an axis of the connector form an angle of about 90 degrees.

40. A connector as claimed in any one of claims 32 to 39, wherein four lobes are provided for each coil of the helical path.

41. A connector as claimed in any one of claims 32 to 40, wherein the helical path has a pitch that is substantially the same as the pitch of a helical feature on the respiratory conduit.

42. A connector as claimed in any one of claims 32 to 41, wherein the one or more protrusions are configured such that a torque required to wind the conduit onto the connector is less than a torque required to unwind the conduit from the connector.

43. A connector as claimed in any one of claims 25 to 42, wherein the first part of the connector is rotatably connected to the second part.

44. A connector as claimed in any one of claims 25 to 43, wherein a first end of the retention element is engaged with the connector.

45. A connector as claimed in any one of claims 25 to 44, wherein the retention element comprises a sleeve.

46. A connector for a respiratory support system, comprising a first part configured to engage a portion of a respiratory conduit, and a second part configured to engage with another connector, the first and second parts together defining an internal lumen for the passage of gas through the first and second parts;
wherein the first part comprises one or more protrusions that form a plurality of lobes aligned along a substantially helical path; and
wherein each lobe comprises a leading portion at a leading end of the lobe and a trailing portion at a trailing end of the lobe, the trailing portion having a steeper gradient with respect to a surface of the first part than the leading portion.

47. A connector as claimed in claim 46, wherein, during assembly of the first part of the connector with the respiratory conduit, the leading portion of each, lobe engages the wall of the conduit before the respective trailing portion.

48. A connector as claimed in claim 46 or 47, wherein the one or more protrusions form a continuous or discontinuous thread.

49. A connector as claimed in any one of claims 46 to 48, wherein the height of each lobe, from the base of the lobe to the top of the lobe, varies along the lobe from the leading portion to the trailing portion.

50. A connector as claimed in any one of claims 46 to 49, wherein the lobes are formed by a single helical protrusion.

51. A connector as claimed in any one of claims 46 to 49, wherein the lobes are formed by a plurality of discrete protrusions.

52. A connector as claimed in any one of claims 46 to 51, wherein a maximum height of each lobe is at a point intermediate the respective leading and trailing ends.

53. A connector as claimed in claim 52, wherein the maximum height of each lobe is substantially the same for each lobe.

54. A connector as claimed in claim 52, wherein the maximum height of each lobe varies between lobes.

55. A connector as claimed in any one of claims 46 to 54, wherein a maximum height of each lobe is at a point closer to the leading end of said lobe than the trailing end of the lobe.

56. A connector as claimed in any one of claims 46 to 55, wherein side edges of each lobe are fileted.

57. A connector as claimed in any one of claims 46 to 56, wherein the leading end of each lobe, the trailing end of each lobe, and an axis of the connector form an angle of about 90 degrees.

58. A connector as claimed in any one of claims 46 to 57, wherein four lobes are provided for each coil of the helical path.

59. A connector as claimed in any one of claims 46 to 58, wherein the helical path has a pitch that is substantially the same as the pitch of a helical feature on the respiratory conduit.

60. A connector as claimed in any one of claims 46 to 59, wherein the one or more protrusions are configured such that a torque required to wind the conduit onto the connector is less than a torque required to unwind the conduit from the connector.

61. A connector as claimed in any one of claims 46 to 60, further comprising a retention element, wherein the retention element defines an outer wall spaced outwards from the first part of the connector and the lobes thereon.

62. A connector as claimed in claim 61, wherein a distance between a maximum height of the lobes and an inner surface of the outer wall is selected to inhibit or prevent disconnection of the respiratory conduit from the connector under a generally axial force.

63. A connector as claimed in claim 62, wherein the distance between a surface of the protrusion and an inner surface of the outer wall is selected such that, upon application of an axial force to the conduit that is above a threshold force, the wall of the conduit will tear while the second part of the connector remains engaged with the conduit.

64. A connector as claimed in claim 62 or 63, wherein at least one distance between a surface of the protrusion and an inner surface of the outer wall is less than a maximum wall thickness of the respiratory conduit.

65. A connector as claimed in any one of claims 62 to 64, wherein, in a seated position of the conduit within the connector, the conduit wall is spaced inwards from the inner surface of the outer wall.

66. A connector as claimed in any one of claims 46 to 65, wherein the first part of the connector is rotatably connected to the second part.

67. A connector as claimed in any one of claims 46 to 66, wherein a first end of the retention element is engaged with the connector.

68. A connector as claimed in any one of claims 46 to 67, wherein the retention element comprises a sleeve.

69. A patient interface assembly for a respiratory support system, comprising:
a patient interface;
a respiratory conduit connected to the patient interface; and
a connector as claimed in any one of claims 46 to 68;
wherein the first part of the connector engages an end portion of the respiratory conduit.

70. A patient interface assembly, as claimed in claim 69, wherein a wall of the conduit comprises a flexible film.

71. A patient interface assembly, as claimed in claim 70, wherein the flexible film is breathable.

72. A patient interface assembly, as claimed in any one of claim 70 or 71, wherein the lobes engage the flexible film.

73. A patient interface assembly, as claimed in claim 72, wherein engagement of the lobes causes deflection of the film.

74. A patient interface assembly, as claimed in any one of claims 69 to 73, wherein a wall of the conduit comprises a helical bead.

75. A patient interface assembly, as claimed in claim 74, wherein the connector is configured such that bead is seated between the successive coils of the helical path formed buy the projection(s).

76. A connector for a respiratory support system, comprising a first part configured to engage a portion of a respiratory conduit, and second part configured to engage with another connector, the first and second parts together defining an internal lumen for the passage of gas through the first and second parts;
wherein the first part comprises one or more protrusions shaped to engage an internal surface of a wall of said portion of the respiratory conduit and wherein the protrusions are shaped to minimise stress concentrations in regions of the conduit wall proximate the protrusions upon engagement of the connector with the conduit.

77. A connector as claimed in claim 76, wherein the protrusion(s) forms a plurality of lobes aligned along a substantially helical path; and
wherein each lobe comprises a leading portion at a leading end of the lobe and a trailing portion at a trailing end of the lobe, the trailing portion having a steeper gradient with respect to a surface of the first part than the leading portion.

78. A connector as claimed in claim 77, wherein, during assembly of the first part of the connector with the respiratory conduit, the leading portion of each, lobe engages the wall of the conduit before the respective trailing portion.

79. A connector as claimed in claim 77 or 78, wherein the lobes are formed by a single helical protrusion

80. A connector as claimed in claim 77 or 78, wherein the lobes are formed by a plurality of discrete protrusions.

81. A connector as claimed in any one of claims 77 to 80, wherein a maximum height of each lobe is at a point closer to the trailing end of said lobe than the leading end of the lobe.

82. A connector as claimed in any one of claims 77 to 81, wherein the leading end of each lobe, the trailing end of each lobe, and an axis of the connector form an angle of about 90 degrees.

83. A connector as claimed in any one of claims 77 to 82, wherein four lobes are provided for each coil of the helical path.

84. A connector as claimed in any one of claims 77 to 83, wherein the helical path has a pitch that is substantially the same as the pitch of a helical feature on the respiratory conduit.

85. A connector as claimed in any one of claims 76 to 84, wherein the one or more protrusions are configured such that a torque required to wind the conduit onto the connector is less than a torque required to unwind the conduit from the connector.

86. A patient interface assembly for a respiratory support system comprising:
a patient interface;
a respiratory conduit connected to the patient interface; and
a connector for a respiratory support system
wherein the connector comprises:
a first part configured to engage an end portion of the respiratory conduit;
a second part configured to engage with another component; and
a retention element defining an outer wall;
together, the first and second parts of the connector define an internal lumen for the passage of gas through the first and second parts;
wherein the first part of the connector comprises a wall that is spaced inwards from the outer wall, thereby defining a cavity between said wall and the outer wall to receive the end portion of the respiratory conduit, the wall of the first part having one or more outwardly-projecting protrusions configured to engage the respiratory conduit; and
wherein at least one distance between a surface of the protrusion(s) and an inner surface of the outer wall is less than a maximum wall thickness of the end portion of the respiratory conduit.

87. A patient interface assembly as claimed in claim 86, wherein the closest distance between the, or each, protrusion and the inner surface of the outer wall is less than a bead size of the conduit.

88. A patient interface assembly as claimed in claim 86 or 87, wherein at least one distance between an outer surface of the wall of the first part and an inner surface of the outer wall is greater than the maximum wall thickness of the portion of the respiratory conduit.

89. A patient interface assembly as claimed in any one of claims 86 to 88, wherein at least one distance between a surface of the, or each, protrusion and an inner surface of the outer wall is greater than the maximum wall thickness of the portion of the respiratory conduit.

90. A patient interface assembly as claimed in any one of claims 86 to 89, wherein the closest distance between the, or each, protrusion and the inner surface of the outer wall is greater than a minimum wall thickness of the respiratory conduit.

91. A patient interface assembly as claimed in any one of claims 86 to 90, wherein, when the conduit is connected with the second part, the conduit wall is spaced inwards from the inner surface of the outer wall.

92. A patient interface assembly as claimed in any one of claims 86 to 91, wherein the protrusion(s) forms a plurality of lobes aligned along a substantially helical path; and
wherein each lobe comprises a leading portion at a leading end of the lobe and a trailing portion at a trailing end of the lobe, the trailing portion having a steeper gradient with respect to a surface of the first part than the leading portion.

93. A patient interface assembly as claimed in claim 92, wherein, during assembly of the first part of the connector with the respiratory conduit, the leading portion of each lobe engages the wall of the conduit before the respective trailing portion.

94. A patient interface assembly as claimed in claim 92 or 93, wherein the lobes are formed by a single helical protrusion.

95. A patient interface assembly as claimed in claim 92 or 94, wherein the lobes are formed by a plurality of discrete protrusions.

96. A patient interface assembly as claimed in any one of claims 92 to 95, wherein a maximum height of each lobe is at a point closer to the trailing end of said lobe than the leading end of the lobe.

97. A patient interface assembly as claimed in any one of claims 92 to 96, wherein the leading end of each lobe, the trailing end of each lobe, and an axis of the connector form an angle of about 90 degrees.

98. A patient interface assembly as claimed in any one of claims 92 to 97, wherein four lobes are provided for each coil of the helical path.

99. A patient interface assembly as claimed in any one of claims 92 to 98, wherein the helical path has a pitch that is substantially the same as the pitch of a helical feature on the respiratory conduit.

100. A patient interface assembly as claimed in any one of claims 86 to 99, wherein the one or more protrusions are configured such that a torque required to wind the conduit onto the connector is less than a torque required to unwind the conduit from the connector.

101. A patient interface assembly as claimed in any one of claims 86 to 100, wherein the first part of the connector is rotatably connected to the second part of the connector.

102. A patient interface assembly as claimed in any one of claims 86 to 101, wherein a first end of the retention element is engaged with the connector.

103. A patient interface assembly as claimed in any one of claims 86 to 102, wherein the retention element comprises a sleeve.

104. A patient interface assembly, as claimed in any one of claims 86 to 103, wherein a wall of the conduit comprises a flexible film.

105. A patient interface assembly, as claimed in claim 104, wherein the flexible film is breathable.

106. A patient interface assembly, as claimed in any one of claims 86 to 105, wherein a wall of the conduit comprises a helical bead.

107. A patient interface assembly as claimed in any one of claims 86 to 106, wherein the at least one distance between a surface of the protrusion and an inner surface of the outer wall is selected to inhibit or prevent disconnection of the respiratory conduit from the connector due to a generally axial force.
